SEQUENCE LISTING (18 sequences)

SEQ ID NO: 1 - DNA, 2520 bp, Bacillus anthracis
ttgtttctacaacgtttgtataatgtctttccttcatatagaacattttctcttccattttctcaagaatggtatttaaaacttgaaatgcttgaattggcttttctgctatcgactcaacttgttttaaattttgatacggtccaaattctaaaccgccttttaaatcgacgatatataaatacgtatgatctggttgtgctgtaataagagatgtcattacattctttaaaaatacggttttccccatacgtgttaaaccacctaaagtcatgtgtggtgttttatcaaaattatgataaattgtttcttctaggcttttccctataggtacaagccattttccttgttcaactaatgtttttgaccatccccacttgttaggtatgtctttatgaaatactcgaatacttaatttatagttatcataatgaatccgaacaggtttatttaacccctcacttacaacatcctcgaccttctgaataattttactcggcatacctacaggtaatgtgtatacatatgttgtgctacgatcatcatcaacttgattttgaaacttgggataatgtagcttatcttctcttttaattgcgattccactcacctcaaaaaagacttgaattttctttttatcatcatcttttcgtttgaacttatcacttactaatgcgtatgttaatgctgctgtaggaactattagtaactctaacataaacatttcccccttaaatatcctataaggatatcattgcactcttttagaatataacgggacgggccttttcttatccccttcctgttgtcctgaccttccacattgtattcctttatagaaatgcaactagaacataacgtagaaggtatagaaacgagcctgtaattgttgtgtacaaggttacacgtggaagccaatatggaacacttctccccattttttctgctgccttcatagcaattactgacaaccctgttgctgtccaaataattaccgcttcccctgcaagtgtcataattactcctcctcttccttttcacgaaaggcaataccttttcttgtaaggaccacatcataacaatccattaaaatttcccagtttaaaacatcttcttcctcaccatataaatcttcttctaacacctgcgacaaactgaaatatcccttatattcttttcggtcataaacttcatggtttttcatgtgatttaaaattgattccgtctctgctcttgatcttgattcattgtacattgaccgtaattcctttgatgggtgaagatatgaagttgtgttcaggtgattatactgccaacgcatataaacctctcccctcttgatgtccttagttccacttggtattcctcgtggtcttgatataggtatatgaactagaaggaagcgttttgcctgtcctgttaaattttttttctaaataaacttaaattgaacaggaatgaaaaattttgtgtggaaagatatatattttaaggaggaggtaatatggagtttaaatgtaggttacggattatttttgccgaaaaagaaataagacaaaaagatttttcaaagacaaccggaatcagccaatcaacattaagtgcacttgtaaacaatacaaaactcccaactttccctactgcatataaaattgctaaagcattaaatatgtacatggaggagatttggattaaagaggaggtggaatgagttgtgtctaaaagaaagaaaaccaacctaagaattgtaaaagaaatttatggtgatcgaaaaatagaagaggtgtttgaagaactttacgaaaagatttatggtttaaaagttaaagttcgaaggaaaacatctgtagaattagtcgctgaaaaacaacaagagaaaaaatatactcaggagtaattacaatgtcaaaacaaccaccattcaaaacattgaatcgcattatcgaaaactattatgatcctaaaaatggattaattgaagtagagcaatttatcataggatgcgaagaggaatcactatttgtattagattacaagggaattatgtatcaaaaaacatctgctcaaaaatatgaccagggttatgaaatcttattacttccttatacagaactacattcaaattctaaacaagatttattgcaactattaaagcgtaaaataattgtttatttcacgtatacagaaaatgatcaacaacaagaagatttcgtcccagatatgggaaaacaaatcttttcatttatgagtcatatgttaaaaggagcacagcaaaataaaagagcaattccagtacgatttattttgatggatattgaagcggttggatttattcctaaacttccaaaatttatgtctggatgtagagggtttaatgtgggtacctgcttatttgtagatgataaattaacacttacatatggttataataaagaacaagtagacaaaatgtataataatagcgtgattgtatccattccataatgaaataaaaaagggtctgctcaagtatgagtagac

SEQ ID NO: 2 - DNA, 1362 bp, Bacillus anthracis
atggagaaagtgaaaaagcaagtcaatcttagtaagattttaatacagtatatttttgcattttcaatagggacgattgcattagttactttttttatgattctattccaagttggagttaacgcagagataatcctaccagcaaattattatgaaaaacagattgagtcgcaaagagatgctatagttaaagcggaacaggaagatgatcaaattttaaaacaatataaatatgctctgtatgatttttcaggaaacgttatacaagggagtattaataaagattatgcaggacaaatatggaataacatacataatgatgaaaaaggtgacagtaagtactactatgcaattatagaacgcgataaagatatttgtgtcattctatatacgttacacgctacttttaaaaatcctattttgcagaagtatgtaccgagtccggaagtttgtgaaattgttctttttattctactatttgttgccgaagtgatatttctatctcgctcttttggtaaatcattgtcaaaggaaatgatgattttgagaaaggtaacagataaaattcaaaaggaagatttagattttcaagtggaacaatctaaaataaaagaagtgaatgaagtattaacttctttagtaaaaatgaaagatgaattaaatcattcgttacgtacacagtggaatatggaaacaaatagaagagagcagattgctgctttggcacatgacattaaaacaccgttaacgatattgagagggaatgcagagctattaaatgaatcaatgctagattcaaatcagaagaaatgtaatgagcatattttgaaaagtattgatgagatggaaatatatatgaaatctttactcgatattacgaaatgtgaggaaggcgctccacttcaattcatgcaaacagatctccgagactttattcataatattatagaagaaagtgcaatttgtgttttaggcaaacagttaaatctaattgaaaatgttcaggacataccaaattttattcctattgacgagaccgctttgaaaagagcaattatgaatataatgatgaatgcaattgaatattctccaacaaagggtgacttgatgttctcagtggaaatgattagtgaaaaactgcaatttattgtagaggactcgggaagagggtttacagaagaggaaatgcattcagctacagagcagttttataggggagataaaagtagaaacgccaaagatcatcatggaatggggttatacattgctaaaagctttgcaaagcaacacggtggaaacctttacttaagtaattcggaaaagctgcatggtgcaaaagttgttttggaaatatcagcgtag

SEQ ID NO: 3 - DNA, 1360 bp, Bacillus anthracis
aatattcttctggattttatgaaggaaccaggtaaaagtgtctttttttctactcatattacctctgatttagataaaattgccgatataattattttaattaatgttgggggaattgtagttaatgaggaaaaagatatattagtagagactcatgcattagtaaaaggtgacaatcggttaattaatgagcaaactaaatctctttttttaaatttgcaccaatctcaatatgggtttgaggggattactcataaaaaagatgatgtgcatcgcctaatgcctgatgtattaatggaaagaccaactatagaagatattatgctttcatacattggaggtaataatcatgttaattaaccttgtattgaaagactttatgttagttaaaaaatattccttaattctactcatttttgcagctgtcgctcctatttatgtttcttcacaaatacagttaaatgatggcggtcttgtagggtttcttataacagtgcttttaacagagcatattttgtttagtacaatatcaaaattcgaggatcagtataaaggagctgctttactttgtgcaattccgtacacacgaaatacatttgtaaaagcaaagtatctctttctctttataatcttcatttgtatctctatcattcgtatcattatatcaattattatgccatcgggtatagaaaaactcagcgtaaatgctcttggaataacatttttaattctaagtattctatttggtatattacttcctattcaatttaaatttggttatgacaaaacaaaattcatatcatttttcactgttttttaataccttttgtcgctcctactcttataagaggagttcaatcgattcacattgattttacaattacatttccattcccgcaaattactcaagactggatgctttgttttatttcactagtcattagtcttgtctcaatgattatatctctaaagatatatgcaaaaaaagatttataagtaatttttaaatataatagggatcattgttaaaggaaatatgtaatacatacgaattcagtactttttaaaatttaatacccttttgcaatgtccctttttcttgattaagtaaattttatacatattgaaataaactaaacctttacaccagtagtctttttaattattttactaagcagaggacgaaatcccatcgctatcaggttaaaaaattcattgcttctcaaaataataaatcggattcctatattatagctatgaaaaggtaaaaaaatcgcttttagagtatttcaacatcttatcttgatggcaatggggagttaactgttaagtttaaaaaagaacataatatgaaaaaaga

SEQ ID NO: 4 - DNA, 21 bp, Artificial Sequence
  Description of Artificial Sequence Synthetic primer
taaggaggaggtaatatggag

SEQ ID NO: 5 - DNA, 21 bp, Artificial Sequence
  Description of Artificial Sequence Synthetic primer
cagtagggaaagttgggagtt

SEQ ID NO: 6 - DNA, 21 bp, Artificial Sequence
  Description of Artificial Sequence Synthetic primer
atggcggtcttgtagggtttc

SEQ ID NO: 7 - DNA, 21 bp, Artificial Sequence
  Description of Artificial Sequence Synthetic primer
aagagcatttacgctgagttt

SEQ ID NO: 8 - DNA, 20 bp, Artificial Sequence
  Description of Artificial Sequence Synthetic oligonucleotide
aatcagccaatcaacattaa

SEQ ID NO: 9 - DNA, 21 bp, Artificial Sequence
  Description of Artificial Sequence Synthetic primer
tcgggaagagggtttacagaa

SEQ ID NO: 10 - DNA, 21 bp, Artificial Sequence
  Description of Artificial Sequence Synthetic primer
aaaggtttccaccgtgttgct

SEQ ID NO: 11 - DNA, 21 bp, Artificial Sequence
  Description of Artificial Sequence Synthetic primer
aaggaccacatcataacaatc

SEQ ID NO: 12 - DNA, 21 bp, Artificial Sequence
  Description of Artificial Sequence Synthetic primer
aacttcatatcttcacccatc

SEQ ID NO: 13 - DNA, 21 bp, Artificial Sequence
  Description of Artificial Sequence Synthetic primer
taacacctgcgacaaactgaa

SEQ ID NO: 14 - DNA, 21 bp, Artificial Sequence
  Description of Artificial Sequence Synthetic primer
caagaccacgaggaataccaa

SEQ ID NO: 15 - DNA, 21 bp, Artificial Sequence
  Description of Artificial Sequence Synthetic primer
acttggtattcctcgtggtct

SEQ ID NO: 16 - DNA, 21 bp, Artificial Sequence
  Description of Artificial Sequence Synthetic primer
cacttaatgttgattggctga

SEQ ID NO: 17 - DNA, 21 bp, Artificial Sequence
  Description of Artificial Sequence Synthetic primer
caggtgattatactgccaacg

SEQ ID NO: 18 - DNA, 21 bp, Artificial Sequence
  Description of Artificial Sequence Synthetic primer
aaacgcttccttctagttcat

(19) United States
(12) Patent Application Publication
(10) Pub. No.: US 2006/0223084 A1
(43) Pub. Date: Oct. 5, 2006

(54) Methods and compositions for detecting Bacillus anthracis

(75) Inventor: David Carlson, Austin, TX (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: BioVeris Corporation

(21) Appl. No.: 11/293,577

(22) Filed: Dec. 5, 2005

Related U.S. Application Data
(60) Provisional application No. 60/632,970, filed on Dec. 6, 2004.

(51) Int. Cl. C12Q 1/68; C12P 19/34

(52) U.S. Cl. 435/6; 435/91.2

(57) ABSTRACT

The present invention relates to methods, compositions, and kits for detecting the presence of B. anthracis and binding partners to B. anthracis. The invention also relates to polynucleotide sequences that are specific for the B. anthracis genome and proteins encoded by those sequences.

The present invention relates to methods, compositions, and kits for detecting the presence of B. anthracis and binding partners to B. anthracis. The invention also relates to polynucleotide sequences that are specific for the B. anthracis genome and proteins encoded by those sequences.

B. anthracis is the causative agent of anthrax, a disease often lethal in humans and animals. This bacterium has a two-stage life cycle consisting of vegetative cells and spores. When a host dies from infection, vegetative cells of B. anthracis are released into the environment. These vegetative cells then sporulate to form infectious spores of B. anthracis. For example, a cow can contract anthrax by ingesting spores in contaminated soil. When the cow dies, vegetative cells are released into the soil where sporulation occurs to form new spores. The cycle repeats when a healthy cow ingests B. anthracis spores while grazing. With adequate soil moisture and pH, B. anthracis spores can germinate, allowing the resulting vegetative cells to replicate before the sporulation process begins again. Water sources and air sources can also be contaminated with B. anthracis spores.

Unlike vegetative cells, B. anthracis spores are very stable in the environment. Some studies have documented that spores can remain viable in soil for 40 to 60 years after deposition (Titball et al., J. App. Bacter. Sympos. 70:9S-18S (1991)). Because of spore stability, the relative ease with which this bacterium can be grown, and the lethal nature of anthrax, B. anthracis has unfortunately become a weapon for bioterrorists. In humans, infection often begins when a healthy individual inhales B. anthracis spores. Fortunately, anthrax is a treatable disease if a correct diagnosis is made early on in infection. Thus, methods that rapidly detect the presence of B. anthracis are important for successful treatment of anthrax in humans as well as for prevention of infection in humans and animals by detecting the bacterium in the environment.

Current detection methods include the use of selective bacterial media, such as heart infusion agar supplemented with polymixin, lysozyme, EDTA, and thallous acetate (PLET medium) (Titball et al., J. App. Bacter. Sympos. 70:9S-18S (1991)). This technique is limited in many ways. First, the length of time required to grow colonies on agar does not permit rapid detection of spores. Second, the method is not particularly sensitive and can require a higher concentration of bacteria in a sample than with more sensitive methods of detection. For example, in sole samples, selective media methods can require more than 3 bacterial spores per gram of soil. Finally, selective media methods are not always specific for growing B. anthracis.

Other current detection methods include immunoassays using polyclonal or monoclonal antibodies and animal testing. Immunoassays detect the presence of anti-B. anthracis antibodies in hosts suspected of being infected. Unfortunately, diagnosis with this method can only be made a few days after clinical signs of illness appear, preventing very early detection. Alternatively, polyclonal or monoclonal antibodies to B. anthracis preparations are used in immunoassays. Though these assays decrease the time needed to detect an infection, they nonetheless suffer from a lack of specificity. Antibodies raised to B. anthracis spores often cross-react with other bacteria in the Bacillus family, including Bacillus cereus. Pre-absorption of the sera with other Bacillus species before use in detecting B. anthracis is used to try to reduce such cross-reactivity. Finally, samples suspected of harboring B. anthracis spores, e.g., soil extracts are tested for the ability to cause disease in guinea pigs or mice. Though this technique improves on the sensitivity of detecting B. anthracis spores, it is both costly and requires enough time for anthrax to develop in the animals.

Today, molecular biology techniques are used to detect B. anthracis. But molecular assays for B. anthracis detection are particularly difficult to design since the B. anthracis genome is highly homologous to that of other species of the same genus. Specifically, the B. anthracis genome is so similar to that of B. cereus and B. thuringiensis that these organisms were proposed to be the same species. Current assays for B. anthracis target plasmids that reside in virulent B. anthracis. However, these plasmids can be lost from the bacterium and can be transferred to other bacteria leading to false negative and false positive results.

Genomic DNA targets commonly used for bacterial identification, such as the 16S ribosomal RNA gene, are inadequate to definitively distinguish the closely related Bacillus cereus from B. anthracis (Sacchi et al., Emer. Infect. Dis. 8:1117-22 (2002)). Other researchers use a different approach to attempt to identify sequences specific to B. anthracis cloning Random Amplification of Polymorphic DNA (“RAPD”) PCR products (U.S. Pat. No. 6,448,016, Rastogi et al.). But examination of these cloned sequences by BLAST analysis shows that they are not specific for B. anthracis. Yet other investigators describe genetic variations in the gyr gene that might be useful for detecting B. anthracis (U.S. Pat. No. 6,087,103, Yamada et al.).

Other nucleic acid-based methods for detecting B. anthracis rely on the detection of anthrax exotoxin genes and/or the polyglutamic capsule genes (Jackson et al., Proc. Natl. Acad. Sci. USA 95:1224-29 (1998)), or the atxA gene (Hartley and Baeumner, Anal. Bioanal. Chem. 376:319-27 (2003)). All of these genes are related to virulence and are located on the two plasmids of anthrax bacteria, pXO1 (174 kbp; toxin) and pXO2 (95 kbp; capsule). Under certain conditions, these plasmids are known to be transferred from B. anthracis to the closely related B. cereus and B. thuringiensis (Ruhfel et al., J. Bact. 157:708-11 (1984)). Yet naturally occurring B. cereus and B. thuringiensis may contain DNA from one or both of these plasmids but not cause anthrax (Beyer et al., J. Appl. Microbiol. 87:229-36 (1999)). Therefore, detection of anthrax based solely on plasmid DNA sequences can give rise to a false-positive result.

A number of attempts have been made to identify chromosomal DNA sequences from B. anthracis that would be suitable for specifically identifying the presence of anthrax-causing bacteria. One of the identified sequences, designated BA813, is a 277 bp long DNA fragment (Patra et al., FEMS Microbiol. 15:223-31 (1996)). Another, vrrA, is a region of sequence variability containing variable repeats (caa tat caa caa) (Anderson et al., J. Bacteriol. 178:377-84 (1996)). Still other putatively specific sequences are described by Rastogi et al. in U.S. Pat. No. 6,448,016. However, none of these sequences are restricted to B. anthracis, again leading to false-positive results.

As discussed above, current assays targeting genes on virulence plasmids and assays that focus on chromosomal sequences can lead to false-positive results, incorrectly indicating the presence of B. anthracis in a sample.

Other investigators have identified single-nucleotide polymorphisms (SNPs) that appear to be specific to B. anthracis. These include SNPs in the DNA gyrase subunit B gene (gyrB) (Yamada et al., U.S. Pat. No. 6,087,104) and in the RNA polymerase subunit B gene (rpoB) (Qi et al, Appl. Environ. Microbiol 67:3720-27 (2001)). However, SNP assays are less robust than assays that detect the presence of polynucleotide sequences, and are generally more costly as well.

Another approach to identifying anthrax-specific chromosomal DNA sequences was described by Radnedge et al. (Appl. Environ. Microbial. 69:2755-64 (2003)). Radnedge used subtractive hybridization techniques to identify genome differences that might be exploited in diagnosis of B. anthracis. Their approach differs from the in silico (computer-based) technique described herein and resulted in the identification of different B. anthracis genomic sequences.

Thus, in one embodiment, the present invention discloses DNA sequences that are present in the B. anthracis genome, but are absent from the closely related B. cereus and B. thuringiensis, and are useful in assays for detecting the presence of B. anthracis.

### SUMMARY OF THE INVENTION

In one embodiment, the invention provides polynucleotide sequences that are specific for B. anthracis. In other embodiments, the invention provides oligonucleotide probes suitable for hybridizing with nucleic acid obtained from B. anthracis. In other embodiments, the invention provides oligonucleotide primers suitable for amplifying nucleic acid sequences present in B. anthracis. In yet other embodiments, the invention provides kits for detecting B. anthracis, the kits comprising at least one of the probes and primers of the invention.

In some embodiments, the invention provides binding partners that specifically recognize B. anthracis.

In some embodiments, B. anthracis-specific sequences according to the invention are used in methods to detect the presence of B. anthracis in a sample. These methods include, but are not limited to, assays that involve amplification of nucleic acid sequences and probe-based assays. In yet another embodiment, the invention provides a method of identifying nucleic acid sequences specific for B. anthracis using computer-based search techniques.

In one embodiment, the invention provides a method for detecting B. anthracis comprising:
(a) providing a sample suspected of containing B. anthracis;
(b) forming a composition comprising nucleic acid from the sample, at least one first primer, and at least one second primer;
(c) amplifying any nucleic acid which the primers in step (b) can amplify; and
(d) detecting B. anthracis by detecting the amplification products of step (c),
   (i) wherein at least one primer comprises a nucleotide fragment that is substantially identical to a portion of any of SEQ ID NO. 1, 2, 3, or their complements and wherein the at least one primer specifically binds to B. anthracis DNA or RNA and not to any of B. cereus, B. thuringiensis, and B. subtilis DNA or RNA; and/or (ii) wherein at least one primer comprises at least 12 contiguous nucleotides that are substantially identical to a portion of SEQ ID NO. 1, 2, 3, or their complements. In certain embodiments, B. anthracis nucleic acid is amplified, but not nucleic acid from B. cereus, B. thuringiensis, B. subtilis, and B. halodurans. In certain embodiments, one or both primers can comprise a detectable label. In some embodiments, the nucleotide sequence of the amplicon is substantially identical to a portion or all of the nucleotide sequence of SEQ ID NO. 1, 2, or 3.

In one embodiment, DNA can be extracted from the sample before the composition of (b) is formed. In another embodiment, the amplification can be performed directly on the sample using, for example, reagents such as Lyse-N-Go™ (Pierce Chemical Co., Rockford, Ill.).

In another embodiment, the invention provides a method for detecting B. anthracis comprising:
(a) providing a sample suspected of containing B. anthracis;
(b) contacting nucleic acid from the sample with at least one probe under conditions favorable for hybridization; and
(c) detecting B. anthracis in the sample based on the hybridization products of step (b),
   (i) wherein at least one probe comprises a nucleotide fragment that is substantially identical to a portion of any of SEQ ID NO. 1, 2, 3, or their complements and wherein the at least one probe specifically binds to B. anthracis DNA or RNA and not to any of B. cereus, B. thuringiensis, and B. subtilis DNA or RNA and/or (ii) wherein at least one probe comprises at least 12 contiguous nucleotides that are substantially identical to a portion of SEQ ID NO. 1, 2, 3, or their complements. In certain embodiments, the probe hybridizes under high stringency conditions with B. anthracis nucleic acid, but does not hybridize under high stringency conditions to nucleic acid from B. cereus, B. thuringienis, B. subtilis, and B. halodurans. In certain embodiments, the probe can comprise a detectable label. In some embodiments, the nucleic acid detected is substantially identical to a portion of the nucleotide sequence of SEQ ID NO. 1, 2, or 3 or the complement of SEQ ID NO. 1, 2, or 3.

Additional objects and advantages of the invention will be set forth in part in the description which follows or can be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed. The accompanying drawings, which are incorporated in and constitute a part of this specification, illustrate embodiments of the invention and together with the description, serve to explain the principles of the invention.

### BRIEF DESCRIPTION OF THE DRAWINGS

FIG. 1 provides the DNA sequence of SEQ ID NO. 1 in the 5′ to 3′ direction.

FIG. 2 provides the DNA sequence of SEQ ID NO. 2 in the 5′ to 3′ direction.

FIG. 3 provides the DNA sequence of SEQ ID NO. 3 in the 5′ to 3′ direction.

FIG. 4 shows an ethidium bromide-stained agarose gel of PCR products generated by amplification of a portion of the B. anthracis genome. DNA isolated from B. anthracis was used as positive control for PCR reactions while DNA from E. coli, Bacillus subtilis, Bacillus cereus, Bacillus thuringiensis, and Bacillus halodurans were used as negative controls for PCR reactions. The agarose gel shows a brightly stained PCR product in the B. anthracis lanes, but no PCR product in the lanes from the other bacterial DNA samples. The primers (SEQ ID NOS. 6 and 7) used in the reactions shown on the left panel of the figure (Panel A) amplified a portion of SEQ ID NO. 3. The primers (SEQ ID NOS. 4 and 5) used in the reactions shown on the right panel of the figure (Panel B) amplified a portion of SEQ ID NO. 1.

FIG. 5 is a graph summarizing a PCR experiment in which one of the PCR primers was labeled with an electrochemiluminescent ruthenium chelate, ruthenium(II)tris-bipyridyl ([Ru(bpy)₃]²⁺). The labeled PCR products were captured on a superparamagnetic bead and analyzed in an automated reader (M1R, BioVeris Corporation). Only B. anthracis DNA generated a signal from [Ru(bpy)₃]²⁺-labeled BA4070 primers in a fashion that is linear on a semilog plot over three logs of DNA input. Negative controls using DNA from E. coli, B. subtilis, or B. cereus did not provide a signal. The graph also provides a least squares fit among the B. anthracis data points.

### DESCRIPTION OF THE INVENTION

I. Sequences

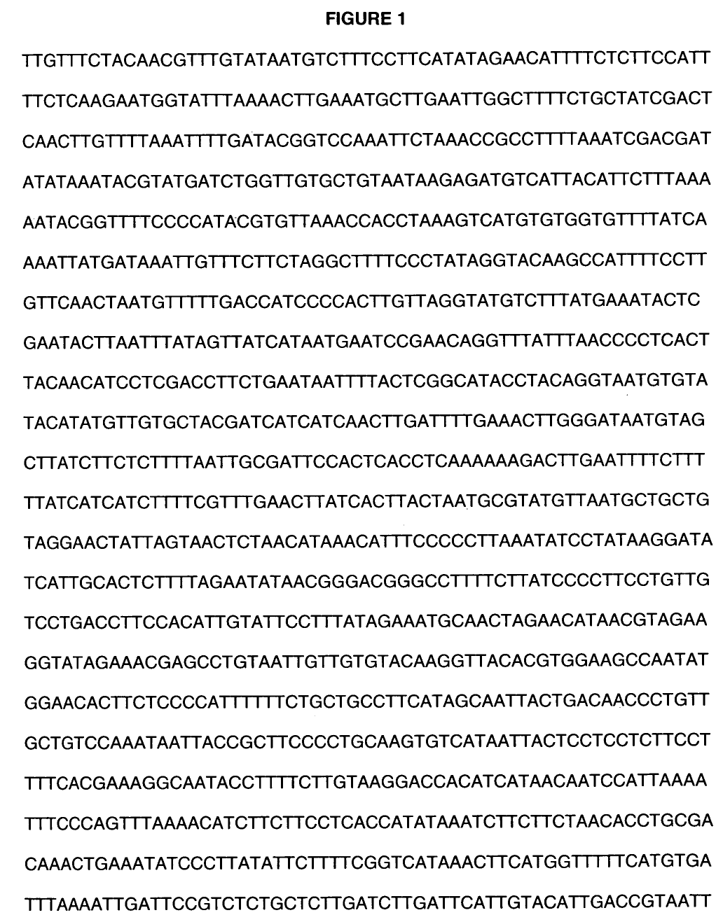

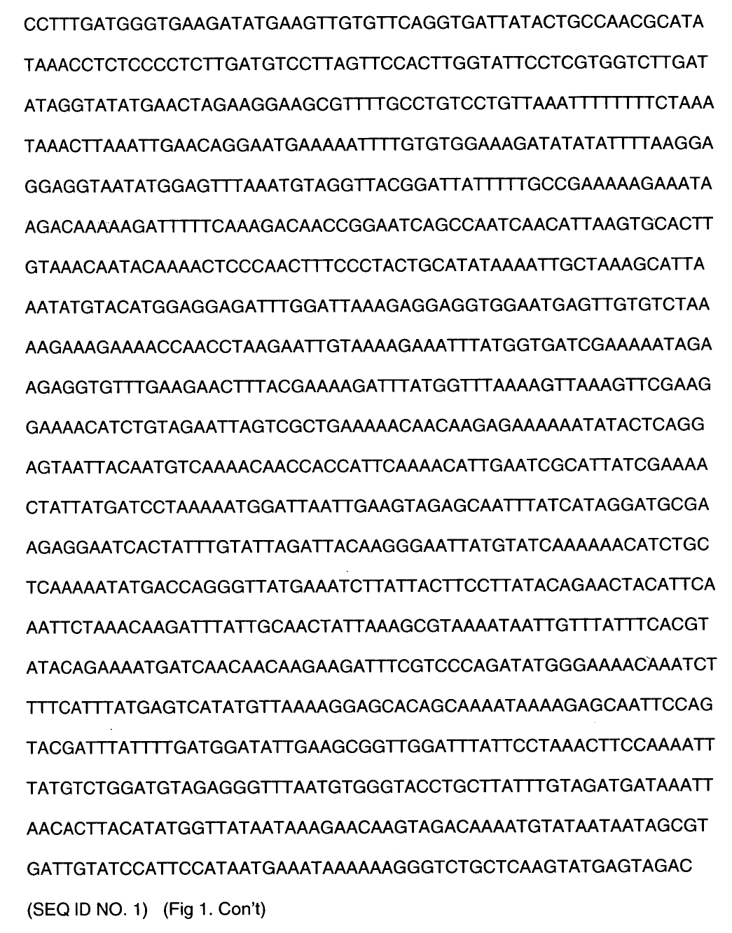

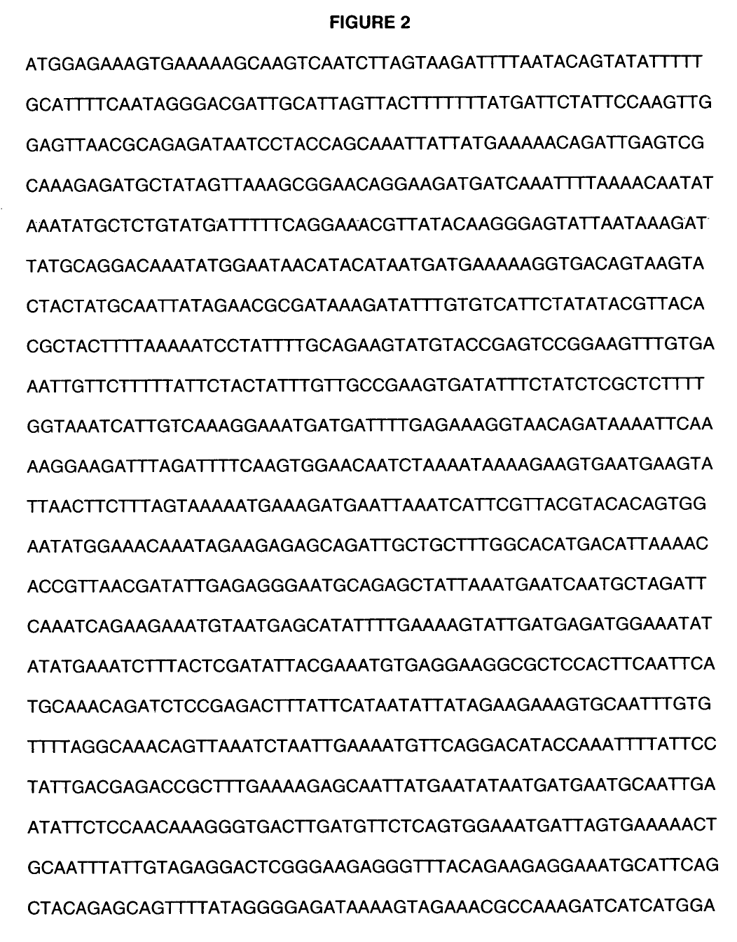

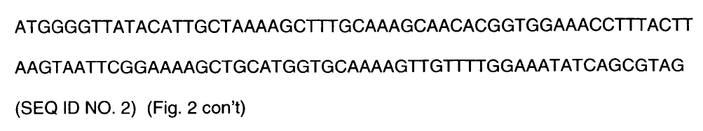

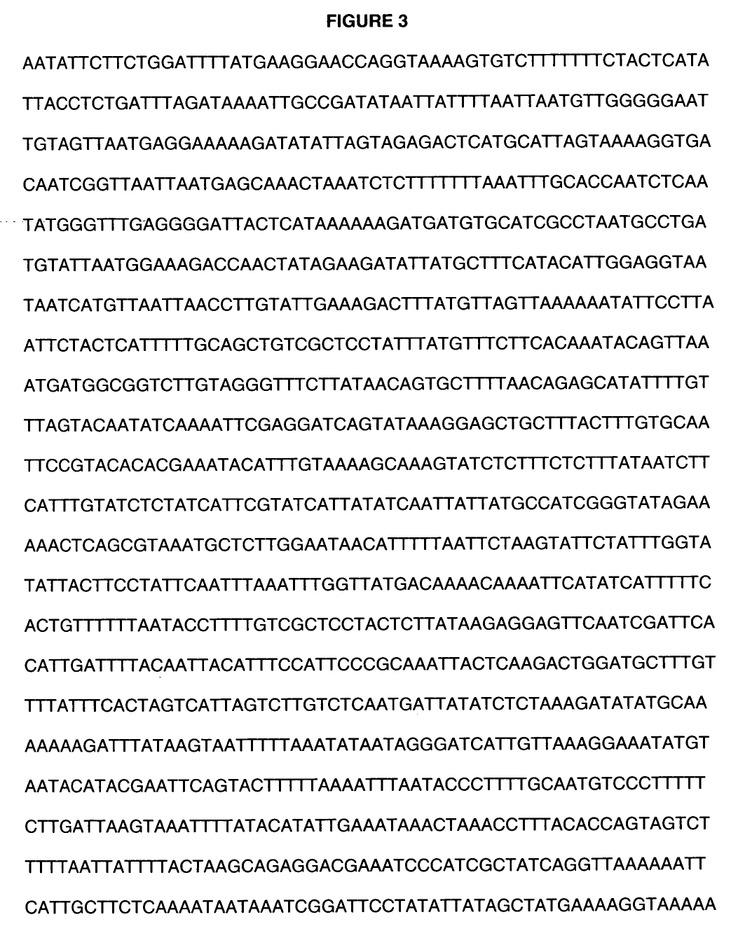

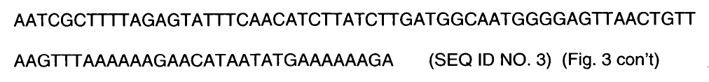

In one embodiment, the invention provides DNA sequences that are specifically found in the genome of B. anthracis and not in the genome of other bacteria, including other species of the Bacillus genus. In another embodiment, the B. anthracis specific DNA sequence is SEQ ID NO. 1, as shown in FIG. 1. In another embodiment, the B. anthracis specific DNA sequence is SEQ ID NO. 2, as shown in FIG. 2. In another embodiment, the B. anthracis specific DNA sequence is SEQ ID NO. 3, as shown in FIG. 3. B. anthracis specific sequences also include portions of the DNA sequences of SEQ ID NO. 1, 2, or 3.

II. Definitions

As used herein, an “amplicon” refers to a nucleotide sequence that is amplified.

As used herein, the abbreviations “bp” and “kbp” refer to “base pair” and “kilo base-pairs.”

The term “binding partner,” as used herein, means a substance that can bind specifically to an analyte of interest. In general, specific binding is characterized by a relatively high affinity and a relatively low to moderate capacity. Nonspecific binding usually has a low affinity with a moderate to high capacity. Typically, binding is considered specific when the affinity constant Ka is higher than about 10⁶ M⁻¹. For example, binding may be considered specific when the affinity constant Ka is higher than about 10⁸ M⁻¹. A higher affinity constant indicates greater affinity, and thus typically greater specificity. For example, antibodies typically bind antigens with an affinity constant in the range of 10⁶ M⁻¹ to 10⁹ M⁻¹ or higher.

The term “antibody,” as used herein, means an immunoglobulin or a part thereof, and encompasses any polypeptide (with or without further modification by sugar moieties (mono and polysaccharides)) comprising an antigen binding site regardless of the source, method of production, or other characteristics. The term includes, for example, polyclonal, monoclonal, monospecific, polyspecific, humanized, single chain, chimeric, synthetic, recombinant, hybrid, mutated, and CDR grafted antibodies as well as fusion proteins. A part of an antibody can include any fragment which can bind antigen, including but not limited to Fab, Fab′, F(ab′)₂, Facb, Fv, ScFv, Fd, the variable region of an antibody heavy chain (V_{H}), and the variable region of an antibody light chain (V_{L}).

As used herein, “substantially identical” means that two polynucleotides hybridize under high stringency conditions.

The term “high stringency” generally refers to hybridization at 5° C. to 15° C. less than the temperature of dissociation. Those skilled in the art will recognize that the temperature of dissociation is dependent upon, among other things, the polynucleotide's base pair composition, the length of the hybridized sequence, probe concentration, salt concentration, and on the solvent used. In some embodiments, high stringency conditions can include hybridization in 50% formamide, 5×SSC, 0.2 μg/μl poly(dA), 0.2 μg/μl human cot1 DNA, and 0.5% SDS, in a humid oven at 42° C. overnight, followed by successive washes in 1×SSC, 0.2% SDS at 55° C. for 5 minutes, followed by washing at 0.1×SSC, 0.2% SDS at 55° C. for 20 minutes. In some embodiments, high stringency conditions include hybridization at 50° C. and 0.1×SSC; overnight incubation at 42° C. in a solution containing 50% formamide, 1×SSC, 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C. In other embodiments, high stringency conditions can also include aqueous hybridization (e.g., free of formamide) in 6×SSC, 1% (SDS) at 65° C. for about 8 hours (or more), followed by one or more washes in 0.2×SSC, 0.1% SDS at 65° C. In one embodiment, high stringency annealing can take place at an annealing temperature that is 5° C. below the melting temperature (Tₘ) of the primer. In general, an approximate primer Tₘ can be calculated by adding 2° C. for each A or T in the primer and 4° C. for each G or C in the primer.

As used herein, a nucleotide sequence “specifically binds to B. anthracis” if it hybridizes under high stringency conditions to nucleic acids from B. anthracis, but not to nucleic acids chosen from:
(i) B. cereus, B. thuringiensis, or B. subtilis;
(ii) B. cereus, B. thuringiensis, B. subtilis, or other members of the Bacillus genus;
(iii) B. cereus, B. thuringiensis, B. subtilis, or B. halodurans; and/or
(iv) B. cereus, B. thuringiensis, B. subtilis, B. halodurans, or other members of the Bacillus genus.

As used herein, the term “polynucleotide of interest” refers to the polynucleotide to be detected or amplified. An amplicon is one example of a polynucleotide of interest. The polynucleotide of interest can be a fragment of a larger nucleic acid sequence.

The term “polynucleotide” refers to a molecule comprising nucleotides or nucleic acid analogs that is greater than 1 nucleotide in length. In one embodiment, a polynucleotide is DNA. In one embodiment, a polynucleotide is RNA. In some embodiments, a polynucleotide can be a recombinant polynucleotide, produced by recombinant methods such as, for example, cloning and expression in a host cell. In some embodiments, a polynucleotide can be an isolated polynucleotide that is substantially free of contaminants. Isolated polynucleotides can be prepared by several methods, including but not limited to, recombinant methods and chemical synthesis. References to polynucleotides include oligonucleotides.

The term “primer” refers to an oligonucleotide that is capable of hybridizing to a target nucleic acid sequence and allowing the synthesis of a complementary strand. Bases in an oligonucleotide primer can be joined by a phosphodiester bond or by a linkage other than a phosphodiester bond, so long as the linkage does not prevent hybridization to a part of the target nucleic acid sequence. For example, oligonucleotide primers can have constituent bases joined by peptide bonds rather than phosphodiester linkages. In some embodiments, a primer can be prepared to be substantially identical to a target nucleic acid sequence.

The term “oligonucleotide” refers to a molecule comprising nucleotides or nucleic acid analogs that is less than 100 nucleotides in length.

A “sample” refers to any substance suspected of containing B. anthracis organisms or spores. A sample also refers to any substance suspected of containing B. anthracis nucleic acid.

As used herein, a “detectable label” refers to moieties that can be attached to an oligomer or polymer to thereby render the oligomer or polymer detectable by an instrument or method.

The term “ECL moiety” refers to an electrochemiluminescent moiety, which is any compound that can be induced to repeatedly emit electromagnetic radiation by exposure to an electrical energy source. Some ECL moieties emit electromagnetic radiation is the visible spectrum while other might emit other types of electromagnetic radiation, such as infrared or ultraviolet light, X-rays, microwaves, etc. Use of the terms “electrochemiluminescence”, “electrochemiluminescent”, “electrochemiluminesce”, “luminescence”, “luminescent” and “luminesce” in connection with the present invention does not require that the emission be light, but admits of the emission being such other forms of electromagnetic radiation.

A “probe,” as used herein, refers to a “nucleic acid” probe or to a “nucleic acid analog” probe that binds to a structure or target of interest.

As used herein, a “nucleic acid” refers to a nucleotide sequence-containing oligomer, polymer, or polymer segment, having a backbone formed solely from naturally occurring nucleotides or unmodified nucleotides.

As used herein, a “modified nucleic acid” means an oligomer, polymer, or polymer segment composed of at least one modified nucleotide, or subunits derived directly from a modification of nucleotides.

The term “nucleic acid analog” refers to synthetic molecules that can bind to a nucleic acid. For example, a nucleic acid analog can be comprised of ribo or deoxyribo nucleotides, modified nucleotides, and/or nucleotide analogs. For example, a nucleic acid analog can comprise a detectable label. The term “nucleotide analog” refers to a synthetic moiety that can be used in place of a natural nucleotide or a modified nucleotide. Nucleic acid analogs can be, but are not limited to, peptide nucleic acids (PNAs), locked nucleic acids (LNAs), or any derivatized form of a nucleic acid.

The term “nucleoside triphosphate” or “nucleotide” refers to a nitrogenous base such as a purine or a pyrimidine that can be covalently bound to a sugar molecule such as ribose or deoxyribose that can be covalently bound to 3 phosphate groups. Nucleoside triphosphates can encompass both ribonucleoside and deoxyribonucleoside triphosphates. A nucleoside triphosphate can be used as a building block to form a polynucleotide. Nitrogenous bases can be, but are not limited to, cytosine, guanine, adenine, thymidine, uracil, and inosine. Nucleoside triphosphates can be, but are not limited to, deoxyadenosine triphosphate (dATP), deoxycytidine triphosphate (dCTP), deoxyguanosine triphosphate (dGTP), deoxythymidine triphosphate (dTTP), deoxyuracil triphosphate (dUTP), and deoxyinosine triphosphate (dITP), 7-deaza-dGTP, 2-aza-dATP, and N4-methyl-dCTP.

The term “modified nucleotide” refers to a nucleotide that has been chemically modified. Non-limiting examples of modified nucleotides can be: 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine).

As used herein, “peptide nucleic acid” or “PNA” means any oligomer or polymer comprising at least one or more PNA subunits (residues), including, but not limited to, any of the oligomer or polymer segments referred to or claimed as peptide nucleic acids in U.S. Pat. Nos. 5,539,082; 5,527,675; 5,623,049; 5,714,331; 5,718,262; 5,736,336; 5,773,571; 5,766,855; 5,786,461; 5,837,459; 5,891,625; 5,972,610; 5,986,053; 6,107,470; 6,201,103; 6,228,982 and 6,357,163. The term PNA also applies to any oligomer or polymer segment comprising one or more subunits of those nucleic acid mimics described in the following publications: Lagriffoul et al., Bioorg. Med. Chem. Lett. 4:1081-82 (1994); Petersen et al., Bioorg. Med. Chem. Lett. 6:793-96 (1996); Diderichsen et al., Tett. Lett. 37:475-78 (1996); Fujii et al., Bioorg. Med. Chem. Lett. 7:637-40 (1997); Jordan et al., Bioorg. Med. Chem. Lett. 7:687-90 (1997); Krotz et al., Tett. Lett. 36:6941-44 (1995); Lagriffoul et al., Bioorg. Med. Chem. Lett. 4:1081-82 (1994); Diederichsen, U., Bioorg. Med. Chem. Lett. 7:1743-46 (1997); Lowe et al., J. Chem. Soc. Perkin Trans. 11:539-46 (1997); Lowe et al., J. Chem. Soc. Perkin Trans. 11:547-54 (1997); Lowe et al., J. Chem. Soc. Perkin Trans. 11:555-60 (1997); Howarth et al., J. Org. Chem. 62:5441-50 (1997); Altmann, K-H et al., Bioorg. Med. Chem. Lett. 7:1119-22 (1997); Diederichsen, U., Bioorganic & Med. Chem. Lett. 8:165-168 (1998); Diederichsen et al., Angew. Chem. Int. Ed. 37:302-305 (1998); Cantin et al., Tett. Lett. 38:4211-4214 (1997); Ciapetti et al., Tetrahedron 53:1167-76 (1997); Lagriffoule et al., Chem. Eur. J. 3:912-919 (1997); Kumar et al., Organic Letters 3:1269-72 (2001); and the Peptide-Based Nucleic Acid Mimics (PENAMS) of Shah et al. as disclosed in WO96/04000.

As used herein, the term “phosphorothioate linkages” means a nucleic acid that comprises the modified internucleotide linkages with phosphorothioate rather than the canonical phosphodiesters. These types of modifications reduce the probability of degradation of oligomer catalyzed by a nuclease and can be used in this invention.

As used herein, the term “locked nucleic acid” or “LNA” means an oligomer or polymer comprising at least one or more LNA subunits. As used herein, the term “LNA subunit” means a ribonucleotide containing a methylene bridge that connects the 2′-oxygen of the ribose with the 4′-carbon. See generally, Kurreck, Eur. J. Biochem. 270:1628-44 (2003).

As used herein, the term “bind” is synonymous with “hybridize.” When two molecules hybridize, they form a combination of the two molecules through one or more types of chemical bonds or through complementary base pairing.

As used herein, the term “complementary” refers to nucleobases that can hybridize to each other. For example, adenine is complementary to thymine and cytosine is complementary to guanine.

III. Compositions of the Invention

A. Polynucleotide Compositions

Polynucleotide compositions of the invention include B. anthracis-specific sequences. In certain embodiments, polynucleotides include SEQ ID NO. 1, 2, or 3. And, in other embodiments, polynucleotides include fragments of SEQ ID NO: 1, 2, or 3, wherein the fragment:
(i) comprises a sequence substantially identical to a portion of any of SEQ ID NO. 1, 2, 3, or their complements and specifically binds to B. anthracis DNA or RNA or
(ii) comprises at least 12 contiguous nucleotides that are substantially identical to a portion of SEQ ID NO. 1, 2, 3, or their complements.
   The polynucleotide compositions of the invention may be used as both primers and probes to detect B. anthracis, and amplicons may be produced by using the primers. In certain embodiments of the invention the fragment is at least 10 bp long, at least 12 bp long, at least 15 bp long, at least 20 bp long, at least 30 bp long, at least 40 bp long, at least 50 bp long, at least 60 bp long, at least 70 bp long, at least 80 bp long, at least 90 bp long, at least 100 bp long, at least 200 bp long, at least 300 bp long, at least 400 bp long, at least 500 bp long, at least 600 bp long, at least 700 bp long, at least 800 bp long, at least 900 bp long, at least 1000 bp long, at least 1360 bp long, at least 1362 bp long, at least 1500 bp long, at least 2000 bp long, at least 2500 bp long, at least 2520 bp long, or any length in between.

In some embodiments, B. anthracis-specific sequences can comprise sequences containing mutations of sequences found in SEQ ID NO. 1, 2, or 3. Mutations include, but are not limited to, substitutions, additions, and deletions of one or more base pairs. In some embodiments, a polynucleotide of the invention can contain 0 to 5, 0 to 10, 0 to 20, 0 to 30, 0 to 40, 0 to 50, 0 to 60, 0 to 70, 0 to 80, or 0 to 90 nucleotide additions, deletions, or substitutions of nucleotide bases in comparison to sequences found in SEQ ID NO. 1, 2, or 3, or their complements. In some embodiments, a polynucleotide of the invention can have 80%, 85%, 89%, 90%, 95%, 96%, 97%, 98%, or 99% sequence identity to sequences found in SEQ ID NO. 1, 2, 3, or their complements. In embodiments where the polynucleotide of the invention is RNA, the thymidine nucleotides in SEQ ID NOS. 1, 2, and 3 will be considered equivalent to the uracil nucleotides in RNA for the purposes of identifying mutations in a polynucleotide sequence.

In certain embodiments, the invention also includes polynucleotides comprising the polynucleotides described earlier in this section. In one embodiment, the invention provides an expression vector comprising B. anthracis-specific sequences found in one or more of SEQ ID NOs. 1, 2, and 3. An expression vector comprises a promoter operably linked to the sequence to be expressed and a replication origin. In one embodiment, the expression vector further comprises other genetic regulatory elements, such as an enhancer, to further regulate expression. In one embodiment, the invention provides an cloning vector comprising B. anthracis-specific sequences found in one or more of SEQ ID NOs. 1, 2, and 3. Cloning vectors comprise a sequence regulating reproduction of the cloning vector such as, for example, an origin of replication. Vectors include, but are not limited to, plasmids, YACS, and artificial chromosomes. The invention also provides cells comprising these expression vectors or cloning vectors.

B. Primers

In some embodiments, each oligonucleotide of a pair of oligonucleotide primers comprises at least 12 contiguous nucleotides of SEQ ID NO. 1, 2, or 3 or at least 12 contiguous nucleotides complementary to SEQ ID NO. 1, 2, or 3 and amplifies B. anthracis-specific nucleic acid. In certain embodiments, an amplicon generated by using a primer pair may span the border of SEQ ID NO 1, 2, or 3 and surrounding sequences of the B. anthracis genome. In certain of these embodiments, a primer pair is used wherein the first primer is substantially identical to a portion of SEQ ID NO. 1, 2, or 3 and specifically binds to B. anthracis, while the second primer is not. Specific amplification of the B. anthracis DNA can still be possible, due to the specificity imparted by the first primer.

A primer represents a 5′ terminus of one strand of the resulting extension product. A primer that is complementary at its 3′ terminus to the sequence of interest on the template strand can be extended using a polymerase to synthesize a sequence complementary to the template. Modifications to the 3′ end can affect the ability of an oligonucleotide to function as primer. An example of such a modification is the incorporation of a Locked Nucleic Acid (LNA) nucleotide, which can enhance the specificity of the primer (Latorra et al., Hum. Mutat. 22:79-85 (2003)). The length of the primer can be adjusted depending upon the particular application, but 15-30 base pairs is a common size. In some embodiments of the invention, the primer can be from 12 to 60 nucleotides in length. In other embodiments, the primer can be from 10 to 30 nucleotides in length. Primers can be used in pairs to amplify the nucleic acid sequencer that falls between the two primer binding sites on the sequence of interest.

A primer need not be a perfect complement for successful hybridization and amplification to take place. For example, primers of 15-60 nucleotides in length can have at least 12 bases of contiguous identity to SEQ ID NO. 1, 2, or 3 or its complement. One skilled in the art will recognize that the optimum amount of identity between the primer and the target for successful amplification depends on a variety of readily controlled features including the annealing temperature, the salt concentration, primer length, and the location of mismatches, if any. If the primer is an imperfect complement, an extension product will result that incorporates the primer sequence, and during a later cycle, the complement to the primer sequence will be incorporated into the template sequence. In one embodiment, a primer can incorporate any nucleic acid base, any modified nucleic acid, or nucleic acid analog so that the primer extension product will incorporate these features to permit separation and detection of the primer extension product. In one embodiment, when the amplification product is formed, that amplification product can be detected by the characteristic properties of a detectable label incorporated into the primer, for example [Ru(bpy)₃]²⁺ or [Ru(sulfo-bpy)₂ bpy]²⁺. Alternatively, one or both primers can incorporate a molecule, e.g., biotin, that renders the primer detectable using a labeled binding partner, e.g., avidin.

In some embodiments, one primer will be partly or completely identical to at least 12 consecutive nucleotides of SEQ ID. NOS. 1, 2, or 3 and the other primer will be complementary to at least 12 consecutive nucleotides of SEQ ID NO. 1, 2, or 3. In some embodiments, both primer sequences are derived from SEQ ID NO. 1. In another embodiment, both primer sequences are derived from SEQ ID NO. 2. In another embodiment, both primer sequences are derived from SEQ ID NO. 3. In some embodiments, if the test substance contains B. anthracis nucleic acid, thousands to millions of copies of the amplicon will be synthesized. In some embodiments, if the test substance does not contain B. anthracis nucleic acid, no detectable DNA will be amplified.

In one embodiment, a detectable label can be directly or indirectly attached to a primer. For example, a detectable label can be indirectly attached to a primer or to a probe using a linker. A detectable label can be, for example, a fluorophore, a chromophore, a spin label, a radioisotope, an enzyme, Quantum Dot, beads, aminohexyl, pyrene, an antigenic determinant detectable by an antibody, a chemiluminescence moiety, or an electrochemiluminescence moiety (ECL moiety), haptens, luminescent labels, radioactive labels, quantum dots, beads, aminohexyl, pyrene, metal particles, spin labels, and dyes.

Fluorophores that can be used in the method of the present invention include, but are not limited to, IR dyes, Dyomics dyes, phycoerythrine, cascade blue, Oregon green 488, pacific blue, rhodamine derivatives such as rhodamine green, 5(6)-carboxyfluorescein, cyanine dyes (i.e., Cy2, Cy3, Cy 3.5, Cy5, Cy5.5, Cy 7) (diethylamino)coumarin, fluorescein (i.e., FITC), tetramethylrhodamine, lissamine, Texas Red, AMCA, TRITC, bodipy dyes, Alexa dyes, green fluorescent protein (GFP), GFP analogues, reef coral fluorescent proteins (RCFPs), RCFP analogues, and tandem dyes as described in U.S. Pat. Nos. 5,783,673; 5,272,257; and 5,171,843.

Enzyme labels that can be used in the present invention include, but are not limited to, soybean peroxidase, alkaline phosphatase, and horseradish peroxidase.

Radioisotopes that can be used in the present invention include, but are not limited to ³²P and ³⁵S.

Chemiluminescent moieties that can be used in the present invention include, but are not limited to, acridinium, luminol, isoluminol, acridinium esters, acridinedione 1,2-dioxetanes, pyridopyridazines.

Representative ECL moieties are described in ELECTROGENERATED CHEMILUMINESCENCE, Bard, Editor, Marcel Dekker, (2004); Knight, A and Greenway, G. Analyst 119:879-890 1994; and in U.S. Pat. Nos. 5,221,605; 5,591,581; 5,858,676; and 6,808,939. Preparation of primers comprising ECL moieties is well known in the art, as described, for example, in U.S. Pat. No. 6,174,709.

ECL moieties can be transition metals. For example, the ECL moiety can comprise a metal-containing organic compound wherein the metal can be chosen, for example, from ruthenium, osmium, rhenium, iridium, rhodium, platinum, palladium, molybdenum, and technetium. For example, the metal can be ruthenium or osmium. For example, the ECL moiety can be a ruthenium chelate or an osmium chelate. For example, the ECL moiety can comprise bis(2,2′-bipyridyl)ruthenium(II) and tris(2,2′bipyridyl)ruthenium(II). For example, the ECL moiety can be ruthenium (II) tris bipyridine ([Ru(bpy)₃]²⁺). The metal can also be chosen, for example, from rare earth metals, including but not limited to cerium, dysprosium, erbium, europium, gadolinium, holmium, lanthanum, lutetium, neodymium, praseodymium, promethium, terbium, thulium, and ytterbium. For example, the metal can be cerium, europium, terbium, or ytterbium.

Metal-containing ECL moieties can have the formula
M(P)ₘ(L1)ₙ(L2)ₒ(L3)ₚ(L⁴)_{q}(L5)ᵣ(L6)ₛ
wherein M is a metal; P is a polydentate ligand of M; L1, L2, L3, L4, L5 and L6 are ligands of M, each of which can be the same as, or different from, each other; m is an integer equal to or greater than 1; each of n, o, p, q, r and s is an integer equal to or greater than zero; and P, L1, L2, L3, L4, L5 and L6 are of such composition and number that the ECL moiety can be induced to emit electromagnetic radiation and the total number of bonds to M provided by the ligands of M equals the coordination number of M. For example, M can be ruthenium. Alternatively, M can be osmium.

Some examples of the ECL moiety can have one polydentate ligand of M. The ECL moiety can also have more than one polydentate ligand. In examples comprising more than one polydentate ligand of M, the polydentate ligands can be the same or different. Polydentate ligands can be aromatic or aliphatic ligands. Suitable aromatic polydentate ligands can be aromatic heterocyclic ligands and can be nitrogen-containing, such as, for example, bipyridyl, bipyrazyl, terpyridyl, 1,10 phenanthroline, and porphyrins.

Suitable polydentate ligands can be unsubstituted, or substituted by any of a large number of substituents known to the art. Suitable substituents include, but are not limited to, alkyl, substituted alkyl, aryl, substituted aryl, aralkyl, substituted aralkyl, carboxylate, carboxaldehyde, carboxamide, cyano, amino, hydroxy, imino, hydroxycarbonyl, aminocarbonyl, amidine, guanidinium, ureide, maleimide sulfur-containing groups, phosphorus-containing groups, and the carboxylate ester of N-hydroxysuccinimide.

In some embodiments, at least one of L1, L2, L3, L4, L5 and L6 can be a polydentate aromatic heterocyclic ligand. In various embodiments, at least one of these polydentate aromatic heterocyclic ligands can contain nitrogen. Suitable polydentate ligands can be, but are not limited to, bipyridyl, bipyrazyl, terpyridyl, 1,10 phenanthroline, a porphyrin, substituted bipyridyl, substituted bipyrazyl, substituted terpyridyl, substituted 1,10 phenanthroline or a substituted porphyrin. These substituted polydentate ligands can be substituted with an alkyl, substituted alkyl, aryl, substituted aryl, aralkyl, substituted aralkyl, carboxylate, carboxaldehyde, carboxamide, cyano, amino, hydroxy, imino, hydroxycarbonyl, aminocarbonyl, amidine, guanidinium, ureide, maleimide a sulfur-containing group, a phosphorus-containing group or the carboxylate ester of N-hydroxysuccinimide.

Some ECL moieties can contain two bidentate ligands, each of which can be bipyridyl, bipyrazyl, terpyridyl, 1,10 phenanthroline, substituted bipyridyl, substituted bipyrazyl, substituted terpyridyl or substituted 1,10 phenanthroline.

Some ECL moieties can contain three bidentate ligands, each of which can be bipyridyl, bipyrazyl, terpyridyl, 1,10-phenanthroline, substituted bipyridyl, substituted bipyrazyl, substituted terpyridyl or substituted 1,10-phenanthroline. For example, the ECL moiety can comprise ruthenium, two bidentate bipyridyl ligands, and one substituted bidentate bipyridyl ligand. For example, the ECL moiety can contain a tetradentate ligand such as a porphyrin or substituted porphyrin.

In some embodiments, the ECL moiety can have one or more monodentate ligands, a wide variety of which are known to the art. Suitable monodentate ligands can be, for example, carbon monoxide, cyanides, isocyanides, halides, and aliphatic, aromatic and heterocyclic phosphines, amines, stibines, and arsines.

In some embodiments, one or more of the ligands of M can be attached to additional chemical labels, such as, for example, radioactive isotopes, fluorescent components, or additional luminescent ruthenium- or osmium-containing centers.

For example, the ECL moiety can be tris(2,2′-bipyridyl)ruthenium(II) tetrakis(pentafluorophenyl)borate. For example, the ECL moiety can be bis[(4,4′-carbomethoxy)-2,2′-bipyridine]2-[3-(4-methyl-2,2′-bipyridine-4-yl)propyl]-1,3-dioxolane ruthenium (II). For example, the ECL moiety can be bis(2,2′bipyridine) [4-(butan-1-al)-4′-methyl-2,2′-bipyridine]ruthenium (II). For example, the ECL moiety can be bis(2,2′-bipyridine) [4-(4′-methyl-2,2′-bipyridine-4′-yl)-butyric acid]ruthenium (II). For example, the ECL moiety can be (2,2′-bipyridine)[cis-bis(1,2-diphenylphosphino)ethylene]{2-[3-(4-methyl-2,2′-bipyridine-4′-yl)propyl]-1,3-dioxolane}osmium (II). For example, the ECL moiety can be bis(2,2′-bipyridine) [4-(4′-methyl-2,2′-bipyridine)-butylamine]ruthenium (II). For example, the ECL moiety can be bis(2,2′-bipyridine) [1-bromo-4(4′-methyl-2,2′-bipyridine-4-yl)butane]ruthenium (II). For example, the ECL moiety can be bis(2,2′-bipyridine)maleimidohexanoic acid, 4-methyl-2,2′-bipyridine-4′-butylamide ruthenium (II).

For example, the ECL moiety can be [Ru(sulfo-bpy)₂bpy]²⁺ whose structure is 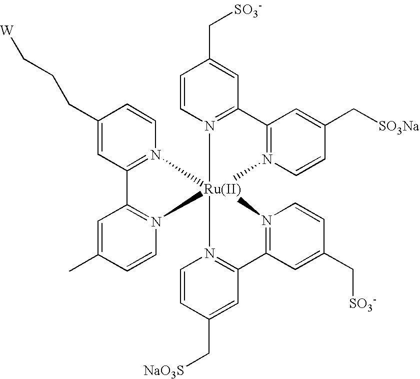
wherein W is a functional group attached to the ECL moiety that can react with a biological material, binding reagent, enzyme substrate or other assay reagent so as to form a covalent linkage such as an NHS ester, an activated carboxyl, an amino group, a hydroxyl group, a carboxyl group, a hydrazide, a maleimide, or a phosphoramidite.

In certain embodiments of the invention, the detectable label can be a molecular beacon (i.e., a conformation-sensitive label attached to a hairpin loop-containing oligonucleotide) as described, for example, in Kostrikis, L. et al., Science 279:1228-29 (1998) and in Tyagi, S. et al., Nat. Biotechnol. 16:49-52 (1998).

In certain embodiments, a primer may be labeled by incorporating a binding moiety into the primer. Binding moieties that can be used in the present invention include biotin and digoxigenin. Biotin, for example, allows another indicator moiety such as a streptavidin coated bead to specifically attach to a probe or to a primer.

C. Amplicons

In certain embodiments, oligonucleotide primers complementary to part of SEQ ID NO. 1, 2, or 3 may be prepared so that a sample nucleotide sequence that falls between the locations where the primers bind is amplified to form an amplicon. In some embodiments, an amplicon includes the sequences of the two primers and the sequence of the nucleic acid that lies between the two primer binding sites. In some embodiments, an amplicon is a detectable portion of SEQ ID NO. 1, 2, or 3. In certain embodiments, the amplicon can be from 50 base pairs to 100,000 base pairs, 50 base pairs to 3000 base pairs, 50 base pairs to 2500 base pairs, 50 base pairs to 2000 base pairs, 50 base pairs to 1500 base pairs, 50 base pairs to 1000 base pairs, 50 base pairs to 500 base pairs, or 50 base pairs to 100 base pairs in length, or any length in between.

In certain embodiments, an amplicon nucleotide sequence or a probe nucleotide sequence can be substantially identical to a detectable portion of a polynucleotide sequence of interest, or its complement, such that the amplicon nucleotide sequence or the probe nucleotide sequence specifically binds to B. anthracis.

In the case of a PCR reaction, for example, high stringency conditions employ hybridization at 64° C. in a 10 mM Tris-HCl pH 8.3, 50 mM KCl, 2 mM MgCl₂ solution. In the case of a blot, such as a Southern blot or a dot blot, for example, high stringency conditions employ either (1) low ionic strength and high temperature for washing, for example, 0.015 M NaCl/0.0015 M sodium citrate/0.1% SDS at 50° C. or (2) a denaturing agent during hybridization such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is the use of 50% formamide, 5×SSC (0.75M NaCl, 0.075 M sodium citrate), 50 mM sodium phosphate (pH 6.8), 0.1% sodium pyrophosphate, 5× Denhardt's solution, sonicated salmon sperm DNA (50 g/ml), 0.1% SDS, and 10% dextran sulfate at 42° C. with washes at 42° C. in 0.2×SSC and 0.1% SDS. High stringency conditions can also include a wash at 65° C. using 0.1×SSC and 0.1% SDS.

An amplicon nucleotide sequence can contain minor additions, deletions, or substitutions of nucleotide bases in comparison to the detectable portion of a polynucleotide sequence of interest. One skilled in the art will recognize that the number of additions, deletions, or substitutions that can be present in an amplicon nucleotide sequence depends on the length and composition of the amplicon. In some embodiments, an amplicon nucleotide sequence can contain 0 to 5, 0 to 10, 0 to 20, 0 to 30, 0 to 40 nucleotide additions, deletions, or substitutions of nucleotide bases in comparison to the detectable portion of a polynucleotide sequence of interest.

D. Probes

A probe nucleotide sequence can also contain minor additions, deletions, or substitutions of nucleotide bases in comparison to the detectable portion of a polynucleotide sequence of interest. One skilled in the art will recognize that the number of additions, deletions, or substitutions that can be present in a probe nucleotide sequence depends on the length and composition of probe. In some embodiments, a probe nucleotide sequence can contain 0 to 5, 0 to 10, 0 to 15, 0 to 20 nucleotide additions, deletions, or substitutions of nucleotide bases in comparison to the detectable portion of a polynucleotide sequence of interest. In some embodiments, the polynucleotide sequence of interest is SEQ ID NO. 1, 2, or 3. In some embodiments, the polynucleotide sequence of interest is a portion of SEQ ID NO. 1, 2, or 3 that is at least 12 base pairs long. In some embodiments, the polynucleotide sequence of interest is a portion of SEQ ID NO. 1, 2, or 3 that is at least 15 base pairs long.

In some embodiments, a probe can comprise a detectable label. In another embodiment, a detectable label can be directly or indirectly attached to a probe as described above for primers. In certain embodiments, a probe may be labeled by incorporating a binding moiety into the probe as described above for primers.

In some embodiments, a probe can be a DNA sequence that is at least 12 consecutive nucleotides of SEQ ID NO. 1, 2, or 3. In various embodiments, a probe can be a DNA sequence that is complementary to at least 12 consecutive nucleotides of SEQ ID NO. 1, 2, or 3. Nucleic acids include, but are not limited to, deoxyribonucleic acid (DNA) and ribonucleic acid (RNA). Non-limiting examples of naturally occurring nucleobases include: adenine, cytosine, guanine, thymine, and uracil. In one embodiment, a probe can be a nucleic acid analog that binds to at least 12 consecutive nucleotides of SEQ ID NO. 1, 2, or 3. In another embodiment, a probe can be a nucleic acid analog that binds to the complement of at least 12 consecutive nucleotides of SEQ ID NO. 1, 2, or 3.

Probes can contain modified nucleotides or nucleic acid analogs. Non-limiting examples of modified nucleotides include: 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9(7-deaza-guanine), N9-(7-deaza-8-aza-guanine) and N8-(7-deaza-8-aza-adenine). Other non-limiting examples of suitable nucleobases include those nucleobases illustrated in FIGS. 2(A) and 2(B) of Buchardt et al. (U.S. Pat. No. 6,357,163). A nucleic acid analog probe can comprise, for example, peptide nucleic acids (PNAs), locked nucleic acids (LNAs), or any derivatized form of a nucleic acid.

In certain embodiments, a probe can contain a mixture of nucleic acids, modified nucleic acids, or nucleic acid analogs. In another embodiment, a probe can comprise nucleic acid segments, modified nucleic acid segments, or nucleic acid analog segments, wherein each segment is separately labeled with a detectable label.

Bases in a probe can be joined by a linkage other than a phosphodiester bond, so long as it does not prevent hybridization. Thus, oligonucleotide probes can have constituent bases joined by peptide bonds rather than phosphodiester linkages. A probe binds to a nucleic acid under certain conditions.

In some embodiments, an oligonucleotide that can be used as a primer or a probe can be 10 to 100 nucleotides long, 10 to 90 nucleotides long, 10 to 70 nucleotides long, 10 to 50 nucleotides long, 10 to 40 nucleotides long, 10 to 30 nucleotides long, or 10 to 20 nucleotides long.

IV. Primer-Based Methods of Detection

In some embodiments, the invention provides methods of detecting B. anthracis that use all or part of the nucleotide sequence of SEQ ID NO. 1, 2, or 3 as targets for amplification. Methods of nucleotide sequence amplification include, but are not limited to, the polymerase chain reaction (PCR), nucleic acid sequence based amplification (NASBA; U.S. Pat. No. 5,409,818), ligase chain reaction (LCR; Wu, D. Y. et al., Genomics 4:560-569 (1989)), transcription mediated amplification (TMA; Kwoh, D. Y. et al., Proc. Natl. Acad. Sci. USA 86:1173-77 (1989)), strand displacement amplification (SDA; Walker et al, Nucleic Acids Res. 20:1691-96 (1992)), self-sustained sequence replication (SSSR; Guatelli, J. C. et al., Proc. Natl. Acad. Sci. USA 87:1874-78 (1990)), and Q beta replicase system (Lizardi, P. M. et al, BioTechnology 6:1197-1202 (1988)).

Techniques for amplifying a DNA sequence are well known in the art. See, e.g., Saiki R. K. et al., Science 230:1350-1354 (1985); Mullis et al., U.S. Pat. No. 4,683,195 and Mullis et al., U.S. Pat. No. 4,683,202. In one embodiment, the two primers are mixed with DNA extracted from a sample and with a DNA polymerase, deoxyribonucleotide triphosphates, buffer, and salts and placed in a thermal cycler instrument in a typical PCR reaction.

Samples include, but are not limited to, soil samples, air samples, water samples, tissue samples from a host, and sputum from a host. Exemplary hosts include, but are not limited to, humans and ungulates, such as cows and sheep. Methods for extracting nucleic acids from such samples are well known in the art. Common methods include treatment of samples with proteolytic enzymes followed by extraction with organic solvents (e.g., phenol, chloroform) and binding to silica in the presence of chaotropic agents (Boom, et al. U.S. Pat. No. 5,234,809). Methods for handling blood specimens and nasal swabs are described, for example, in Rantakokko-Jalava K. and Viljanen, M. K., Clin. Microbiol. Infect. 9:1051-56 (2003). Exemplary methods for preparing DNA from anthrax spores in soil samples is described in Cheun H. I. et al., J. Appl. Microbiol. 95:728-33 (2003).

An amplicon can be detected by a variety of means well known to persons skilled in the art. In one embodiment, amplicons can be detected by gel electrophoresis followed by staining with a DNA-specific stain. Gels suitable for such analysis include, but are not limited to, agarose gels and polyacrylamide gels. Stains include, but are not limited to, ethidium bromide, SYBR® Gold and SYBR® Green (Molecular Probes, Eugene, Oreg.), and silver staining (Bassam B. J. et al., Anal. Biochem. 196:80 (1991)). In certain embodiments, the stain, e.g., ethidium bromide, can be incorporated into the gel.

In certain embodiments, the B. anthracis detections assays of the invention can be modified by adding components that give increased specificity or sensitivity. Such additives include, but are not limited to, bovine serum albumin, dimethyl sulfoxide, betaine, and tetramethylammonium chloride. In some embodiments, components that increase ease in handling can also be added.

In some embodiments of the invention, it is possible to detect the presence of a nucleic acid of interest in a sample by incorporating a labeled primer followed by measurement of the labeled amplification product. In one embodiment, ECL labels can be incorporated into one or both of the PCR primers. For example, the oligodeoxynucleotide primers can be prepared to be sufficiently complementary to a B. anthracis nucleic acid sequence of interest. Primers can be labeled with an amino group introduced during synthesis, or directly during synthesis, using tag NHS and tag phosphoramidite, respectively where the tag is an ECL moiety. In some embodiments, a digoxigenin label is detected through a chemiluminescent reaction.

In another embodiment, a detectable label can be incorporated into the amplified DNA during the synthesis. In another embodiment, a detectable label can be associated with one or both of the oligonucleotide primers. In another embodiment, a detectable label can be bound specifically to newly synthesized DNA. Exemplary techniques for analyzing, staining, and labeling nucleic acids are well known in the art and can be found, for example, in Biren, B, Green, E. D. Klapholz, S., Myers, R. M., and Roskams, J., 1997, Analyzing DNA, Cold Spring Harbor Press, and Kricka, L, (editor), 1995, Nonisotopic Probing, Blotting, and Sequencing, Academic Press.

In certain embodiments, a B. anthracis nucleic acid of interest in a sample can be detected by a method comprising:

(a) providing a sample suspected of containing B. anthracis;

(b) forming a composition comprising nucleic acid from the sample, at least one first primer, and at least one second primer;

(c) amplifying any nucleic acid which the primers in step (b) can amplify; and

(d) detecting B. anthracis by detecting the amplification products of step (c),
(i) wherein at least one primer comprises a nucleotide fragment that is substantially identical to a portion of any of SEQ ID NO. 1, 2, 3, or their complements and wherein the at least one primer specifically binds to B. anthracis DNA or RNA and/or (ii) wherein at least one primer comprises at least 12 contiguous nucleotides that are substantially identical to a portion of SEQ ID NO. 1, 2, 3, or their complements.

In certain embodiments, the method can further comprise incubating the amplicon at a temperature sufficient to denature the amplicon and hybridizing the denatured amplicon with an oligonucleotide that can be or is immobilized on a magnetizable bead before detecting the amplicon. The term “magnetizable” as used herein refers to a property of matter wherein the permeability of the matter differs from that of free space. The term includes paramagnetic and superparamagnetic.

In certain embodiments, the nucleic acid can be amplified using the polymerase chain reaction.

In certain embodiments, sequences present in SEQ ID NO. 1, 2, 3, or their complement may be used to amplify B. anthracis nucleic acids. In one embodiment, B. anthracis sequences can be amplified for cloning into a vector for further cloning or a vector for expression of the a protein encoded by all or part of SEQ ID NO. 1, 2, 3. Methods of cloning are well known in the art. An expression vector comprises a promoter operably linked to the sequence to be expressed and a replication origin. In one embodiment, the expression vector further comprises other genetic regulatory elements, such as an enhancer, to further regulate expression. Vectors include, but are not limited to, plasmids, YACS, and artificial chromosomes.

In one embodiment, the invention provides a method of amplifying Bacillus anthracis nucleic acid comprising:

(a) providing a sample of B. anthracis nucleic acid;

(b) forming a composition comprising the nucleic acid and a primer pair;

(c) amplifying the nucleotide sequence between the first primer of the primer pair and the second primer of the primer pair to form an amplicon; and

(d) detecting the amplicon,

(i) wherein at least one primer of the primer pair comprises a nucleotide fragment that is substantially identical to a portion of any of SEQ ID NO. 1, 2, 3, or their complements and wherein the at least one primer specifically binds to B. anthracis DNA or RNA and/or (ii) wherein at least one primer of the primer pair comprises at least 12 contiguous nucleotides that are substantially identical to a portion of SEQ ID NO. 1, 2, 3, or their complements.

V. Probe-Based Methods of Detection

In addition to amplification-based methods, the B. anthracis specific sequences of the invention can be used as probes for detecting the presence of B. anthracis nucleic acids in a sample by other techniques. For example, the components in a nucleic acid sample can be separated on a gel, transferred to a membrane, and then detected by hybridization with a probe. In certain embodiments, all or part of the sequence of SEQ ID NO. 1, 2, or 3 or the complementary sequence of all or part of the sequence of SEQ ID NO. 1, 2, or 3 can be used as a probe to detect the presence of B. anthracis specific sequences in a nucleic acid sample. In some embodiments, a probe can contain at least 12 bases or more. In certain embodiments, the probe can comprise a detectable label. The probe molecules can be contacted with nucleic acids extracted from a sample under conditions where the probe molecules specifically bind to B. anthracis nucleic acid in the sample, thus making the B. anthracis nucleic acids detectable.

In various embodiments, the probe-based assay is a Southern blot. In certain embodiments, the probe-based assay is a Northern blot. Probe-based assays are well known to those skilled in the art. See, e.g., Southern, E. M., J. Mol. Biol. 98:503-17 (1975); Smith, G. E. and Summers, M. D., Anal. Biochem. 109:123-129 (1980).

Appropriate hybridization conditions can be selected by those skilled in the art based on well-known parameters as exemplified in Ausubel et al. (1995), Current Protocols in Molecular Biology, John Wiley & Sons, sections 2, 4, and 6. Additionally, stringency conditions are described in Sambrook et al. (1989) Molecular Cloning: A Laboratory Manual, 2nd ed., Cold Spring Harbor Press, chapters 7, 9, and 11, for example, 100 mM to 1 M NaCl, and 40° C. to 65° C. One of skill in the art will of course recognize that optimal hybridization conditions vary with the length and composition of the probe.

In other embodiments, the probe can be conjugated to a solid support. Exemplary solid supports include, but are not limited to, glass beads, silica beads, magnetizable beads, multiwell plates, and dipsticks. In other embodiments, the probe further comprises a chemically active group to facilitate attachment to a solid support. Chemically active groups are moieties through which an oligonucleotide can be coupled to a support. In certain embodiments, a chemically active group can be an amino group.

In one embodiment, an oligonucleotide probe comprises all or part of the nucleotide sequence of SEQ ID NO. 1, 2, or 3 or a nucleotide sequence complementary to SEQ ID NO. 1, 2, or 3, wherein the oligonucleotide probe specifically binds to B. anthracis.

In certain embodiments, a B. anthracis nucleic acid of interest in a sample can be detected by a method comprising:
(a) providing a sample suspected of containing B. anthracis;
(b) contacting nucleic acid from the sample with at least one probe under conditions favorable for hybridization; and
(c) detecting B. anthracis in the sample based on the hybridization products of step (b),
   (i) wherein at least one probe comprises a nucleotide fragment that is substantially identical to a portion of any of SEQ ID NO. 1, 2, 3, or their complements and wherein the at least one probe specifically binds to B. anthracis DNA or RNA and/or (ii) wherein at least one probe comprises at least 12 contiguous nucleotides that are substantially identical to a portion of SEQ ID NO. 1, 2, 3, or their complements.

In certain embodiments, the probe is conjugated to a solid support. A solid support can be, for example, a bead or a microtiter plate.

In various embodiments, the probe may be bound to a solid support after hybridization. For example, the probe may contain biotin and/or digoxigenin and be immobilized on a solid support comprising avidin, streptavidin, or an anti-digoxigenin antibody.

In various embodiments, the one or more complementary oligonucleotides are linked to at least one binding moiety via an amino group introduced during synthesis.

VI. Kits

In other embodiments, a kit comprising one or more probes of the invention can be used to practice the methods of the invention for detecting B. anthracis. In other embodiments, a kit comprising one or more of the primers of the invention can be used to practice the methods of the invention for detecting B. anthracis. In yet other embodiments, kits can comprise both the probes and primers of the invention.

In one embodiment, a kit comprises at least one pair of oligonucleotide primers, the primer pair comprising at least 12 contiguous nucleotides of SEQ ID NO. 1, 2, or 3 or at least 12 contiguous nucleotides complementary to SEQ ID NO. 1, 2, or 3 and wherein the primer pair specifically amplifies B. anthracis DNA and does not amplify DNA from B. cereus, B. thuringiensis, and B. subtilis.

In another embodiment, a kit comprises at least one oligonucleotide probe comprising a portion of the nucleotide sequence of SEQ ID NO. 1, 2, or 3 or a portion of a nucleotide sequence complementary to SEQ ID NO. 1, 2, or 3, wherein the oligonucleotide probe specifically binds to B. anthracis.

VII. Binding Partners

In certain embodiments, the invention provides binding partners and methods of making binding partners that bind specifically to B. anthracis proteins, but not to B. cereus, B. thuringiensis, or B. subtilis proteins. Examples of binding partners include complementary polynucleotides (e.g., two DNA sequences which hybridize to each other; two RNA sequences which hybridize to each other; a DNA and an RNA sequence which hybridize to each other), an antibody and an antigen, a receptor and a ligand (e.g., TNF and TNFr-1, CD142 and Factor VIIa, B7-2 and CD28, HIV-1 and CD4, ATR/TEM8 or CMG and the protective antigen moiety of anthrax toxin), an enzyme and a substrate, or a molecule and a binding protein (e.g., vitamin B12 and intrinsic factor, folate and folate binding protein).

In certain embodiments, B. anthracis specific binding partners can be part of a pharmaceutical composition for the treatment or prevention of illness caused by B. anthracis infection. In certain embodiments, the pharmaceutical carrier further comprises a pharmaceutically acceptable carrier. Examples of such carriers include, but are not limited to, sterile liquids, such as water, oils, including petroleum oil, animal oil, vegetable oil, peanut oil, soybean oil, mineral oil, sesame oil, and the like. Saline solutions, aqueous dextrose, and glycerol solutions can also be employed as liquid carriers. Additional examples of carriers include, but are not limited to, liposomes, oil in water emulsions, and or metallic salts, including aluminium salts (such as aluminium hydroxide). Additional suitable pharmaceutical carriers are described in Remington's Pharmaceutical Sciences, 18^{th} Edition.

In certain embodiments, binding partners are produced by recombinant DNA techniques. In certain embodiments, binding partners are produced by enzymatic or chemical cleavage of intact antibodies. In certain embodiments, binding partners are produced by recombinant DNA techniques.

In some embodiments, a binding partner is an antibody. Techniques for producing polyclonal and monoclonal antibodies to protein antigens are well-known in the art. In the field of reverse vaccinology, pathogen genomes can be sequenced and analyzed, looking for potential open reading frames that encode proteins. See generally Capecchi et al., Curr. Issues Mol. Biol. 6:17-28 (2004). During this in silico analysis, these proteins can be analyzed for conserved regions of homology with other known proteins to determine the potential role each protein may play in infection and replication. The Institute for Genomic Research (TIGR) has accomplished these first few steps by sequencing the entire B. anthracis genome and assigning potential functions to each of the putative proteins identified in the genome. The entire bacterial gene or a portion of it can be cloned into an expression vector for expression in a non-pathogenic bacteria such as E. coli. Once the protein is expressed and purified via standard techniques in the art, it can be used as an immunogen to produce polyclonal antibodies or monoclonal antibodies from a host such as a rabbit or mouse. In addition, in vitro selections such as phage display technology can be employed to prepare antibodies for the specific gene products. The resulting antibodies can then be screened via immunoassays, such as an ELISA, to determine whether the antibodies recognize surface antigens on the bacterium. Animal models for bacterial infection or pathogenesis can be used to assess whether the resulting antibodies are neutralizing.

In one embodiment, a method for producing antibodies that bind specifically to at least one B. anthracis protein comprises:

(a) introducing an expression vector comprising all or part of the nucleic acid sequence of at least one of SEQ ID NOS. 1, 2, and 3 into a host cell to express a B. anthracis protein;

(b) isolating the B. anthracis protein; and

(c) immunizing an animal host with a composition comprising the isolated B. anthracis protein so that antibodies are produced.

In certain embodiments, the host cell is a prokaryotic cell, including but not limited to, bacteria. In certain embodiments, the host cell is an archaea cell. In certain embodiments, the host cell is a eukaryotic cell. In certain embodiments, the expression vector is a plasmid, a bacteriophage, a cosmid, a replication-defective adenovirus, an adeno-associated virus, a herpes simplex virus, or a retrovirus. In certain embodiments, the animal host is a mouse or a rabbit.

The following examples are provided for illustrative purposes only and are not intended to limit or restrict the scope of the invention.

### EXAMPLES

### Example 1

### Identification of B. anthracis-specific Genomic Sequences

Segments of B. anthracis DNA that would be suitable targets for diagnostic assays were verified using the BLAST homology search program, available at http://www.ncbi.nlm.nih.gov/BLAST to compare the sequence of a virulent strain (Ames) of B. anthracis to all known bacterial DNA sequences using the “Genbank nr” database of sequences. B. anthracis nucleotide sequences were randomly inserted into BLAST searches of the Genbank nr database. Nucleic acid sequences in B. anthracis that have little identity to gene sequences of other bacteria, and are therefore good targets for detecting B. anthracis, were identified. The bit scores for non-anthracis sequences were less than 100. The bit scores for the identified B. anthracis sequences were 4948, 2658, and 2529 for SEQ ID NOS. 1, 2, and 3, respectively.

These sequences, set forth in SEQ ID NOS. 1, 2, and 3, were segments from the complete DNA sequence of B. anthracis Ames strain in the Genbank database (Accession Number AE016879).

### Example 2

### Detection of B. anthracis-specific Nucleic Acid Sequences Using PCR

The specificity of the identified sequences was demonstrated in a PCR assay. In brief, PCR primer oligonucleotides were designed to give efficient amplification of a portion of SEQ ID NO. 1 or SEQ ID NO. 3. The following primers were used to amplify a portion of the nucleotide sequence in SEQ ID NO. 1:

| | |
|---|---|
| 5′-TAAGGAGGAGGTAATATGGAG | (SEQ ID NO. 4) |
| and | |
| 5′-CAGTAGGGAAAGTTGGGAGTT. | (SEQ ID NO. 5) |

The expected size of this amplicon was 154 bp (FIG. 1, Panel B). The following primers were used to amplify a portion of the nucleotide sequence in SEQ ID NO 3.:

| | |
|---|---|
| 5′-ATGGCGGTCTTGTAGGGTTTC | (SEQ ID NO. 6) |
| and | |
| 5′-AAGAGCATTTACGCTAGAGTTT. | (SEQ ID NO. 7) |

The expected size of this amplicon was 248 base pairs (FIG. 1, Panel A). These primers were used in PCR reactions with B. anthracis DNA as a positive control. Genomic DNA samples from other bacteria, such as E. coli, Bacillus subtilis, Bacillus halodurans, or the closely related Bacillus cereus and Bacillus thuringiensis were used as negative controls.

Each reaction was performed in a 25 μl volume containing 10 mM Tris-HCl pH 8.3, 50 mM KCl, 2 mM MgCl₂, 0.2 mM dNTPs, 0.2 μM each primer, 1 unit AmpliTaq Gold (Applied Biosystems), and 200 pg DNA template. Samples were amplified in a thermal cycler programmed to incubate 7 minutes at 95° C., followed by 35 cycles of 95° C. for 30 seconds, 45° C. for 30 seconds, and 72° C. for 30 seconds, finishing with an extension incubation at 72° C. for 2 minutes. The resulting PCR products were visualized by agarose gel electrophoresis followed by staining in the presence of ethidium bromide.

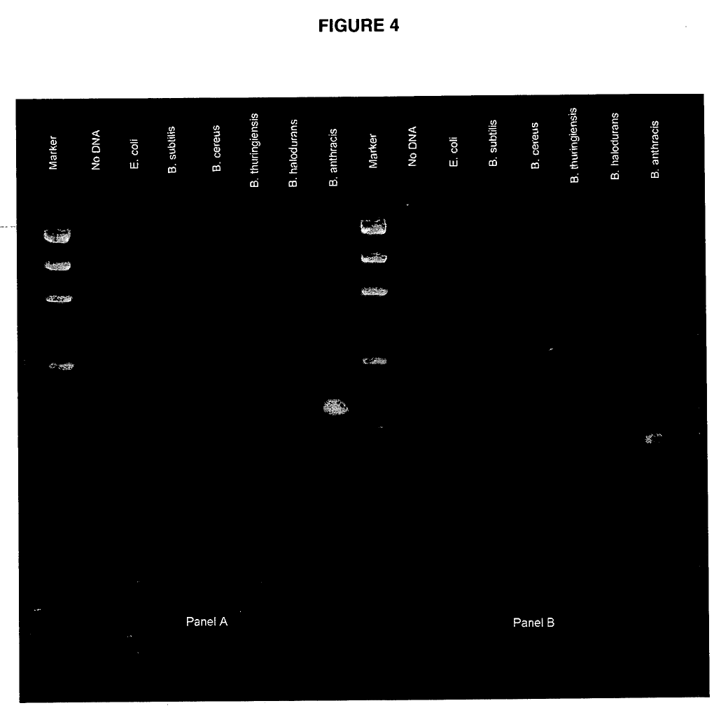

As shown in FIG. 4, the agarose gel shows a brightly stained PCR product of the expected size in the B. anthracis lanes, but no ethidium bromide-stained PCR product in the lanes from other bacterial DNA samples. Size standards in the marker lanes were, from top to bottom, 2000, 1200, 800, 400, 200, and 100 base pairs in length.

### Example 2

### Detection of B. anthracis-specific Nucleic Acid Sequences Using a Combination of PCR and ECL Detection

This example describes the use of ECL-labeled primers to detect B. anthracis, comprising labeling one of the PCR primers with [Ru(bpy)₃]²⁺. The primers set forth in SEQ ID NOS. 4 and 5 were used. The primer SEQ ID NO. 5 was labeled by adding [Ru(bpy)₃]²⁺ to the 5′-end of the oligonucleotide primer during synthesis using a [Ru(bpy)₃]²⁺ phosphoramidite as described in Gudibande et al., U.S. Pat. No. 5,686,244. Amplicons were generated using the PCR protocol described above on DNA isolated from B. anthracis, E. coli, B. subtilis, or B. cereus.

An oligonucleotide was prepared that was complementary to the amplicons obtained by using the above primers in a PCR reaction. This capture oligonucleotide was synthesized to be complementary to a 20-base region between the PCR primers. The capture oligonucleotide sequence was 5′-amineAATCAGCCAATCAACATTAA (SEQ ID NO. 8). The capture oligonucleotide was immobilized on Dynal carboxylated M-270 superparamagnetic beads using 1-ethyl-3-(3-dimethylaminopropyl)carbodiimide hydrochloride reagent according to the manufacturer's instructions (Dynal Biotech, Cat. No. 143.05) to couple the oligonucleotide amino group to the carboxyl group on the beads.

The amplicons were mixed with the immobilized capture oligonucleotide and allowed to hybridize. Specifically, the PCR products in 25 μl were mixed with an equal volume (another 25 μl) of 600 mM NaCl, 20 mM Tris-HCl pH 8.0, 10 mM EDTA, 0.2% TWEEN™ 20 containing 15 μg oligonucleotide beads. The suspension was incubated at 95° C. for 5 minutes, followed by 30 minutes at 45° C. The superparamagnetic beads bearing the amplification product and complementary oligonucleotide complex were then analyzed for bound [Ru(bpy)₃]²⁺ in an automated reader (M1R, BioVeris Corporation).

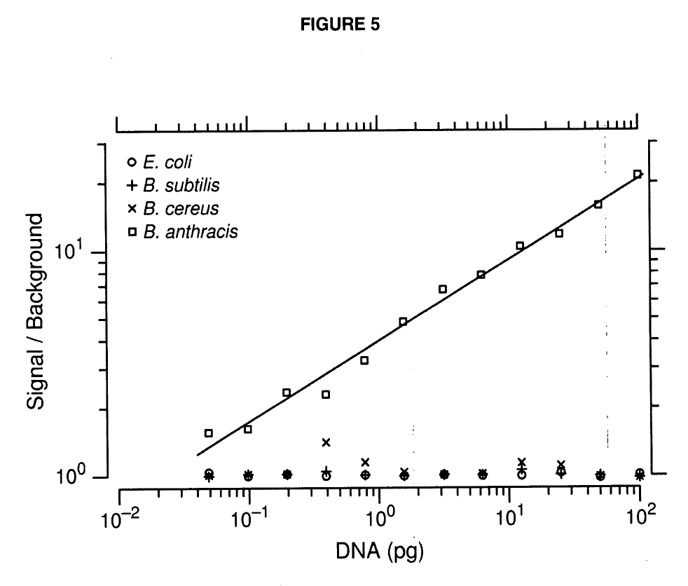

As shown in FIG. 5, only B. anthracis DNA generated a signal from [Ru(bpy)₃]²⁺-labeled primers that is well-fit by a straight line on a semilog plot over three logs of DNA input. Similar results were not obtained with E. coli, B. subtilis, or B. cereus DNA.

### Example 3

### Demonstration of the Generality of the PCR Specificity

To test additional sites throughout the nucleotide sequences of SEQ ID NOS. 1 and 2 for B. anthracis specificity, the following pairs of PCR primers were designed based on these two sequences:

| | |
|---|---|
| 5′-TCGGGAAGAGGGTTTACAGAA | (SEQ ID NO. 9) |
| 5′-AAAGGTTTCCACCGTGTTGCT; | (SEQ ID NO. 10) |
| 5′-AAGGACCACATCATAACAATC | (SEQ ID NO. 11) |
| 5′-AACTTCATATCTTCACCCATC; | (SEQ ID NO. 12) |
| 5′-TAACACCTGCGACAAACTGAA | (SEQ ID NO. 13) |
| 5′-CAAGACCACGAGGAATACCAA; | (SEQ ID NO. 14) |
| 5′-ACTTGGTATTCCTCGTGGTCT | (SEQ ID NO. 15) |
| 5′-CACTTAATGTTGATTGGCTGA; | (SEQ ID NO. 16) |
| and | |
| 5′-CAGGTGATTATACTGCCAACG | (SEQ ID NO. 17) |
| 5′-AAAGGCTTCCTTCTAGTTCAT. | (SEQ ID NO. 18) |

Each of the above primer pairs were used in PCR reactions with B. anthracis DNA (Sterne strain) as a positive control. Genomic DNA samples from forty-three other bacteria, listed in Table 1 below, were used as negative controls. Each reaction was performed in a 25 μl volume containing 10 mM Tris-HCl pH 8.3, 50 mM KCl, 2 mM MgCl₂, 0.2 mM dNTPs, 0.2 μM each primer, 1 unit AmpliTaq Gold® (Applied Biosystems), and 1 ng DNA template. Samples were amplified in a thermal cycler programmed to incubate PCR reactions for 7 minutes at 95° C., followed by 35 cycles of 95° C. for 30 seconds, 45° C. for 30 seconds, and 72° C. for 30 seconds, finishing with an extension incubation at 72° C. for 2 minutes. The resulting PCR products were visualized by agarose gel electrophoresis and ethidium bromide staining. For each of the primer sets, DNA from all of the strains in Table 1 were negative and B. anthracis DNA was positive.

TABLE 1
Species of bacteria tested

| Species | Strain |
|---|---|
| Bacillus anthracis | Sterne 34F2 |
| Bacillus azotoformans | ATCC 29788 |
| Bacillus cereus | ATCC 13061 |
| Bacillus cereus | ATCC 11778 |
| Bacillus cereus | ATCC 49063 |
| Bacillus cereus | ATCC 7004 |
| Bacillus cereus | ATCC 19637 |
| Bacillus cereus | ATCC 7064 |
| Bacillus cereus | ATCC 14579 |
| Bacillus cereus T | BGSC 6A1 |
| Bacillus cereus T-HT | BGSC 6A2 |
| Bacillus circulans | ATCC 9966 |
| Bacillus circulans | ATCC 4513 |
| Bacillus coagulans | ATCC 7050 |
| Bacillus firmus | ATCC 14575 |
| Bacillus halodurans | ATCC BAA-125 |
| Bacillus lichenformis | ATCC 14580 |
| Bacillus megaterium | ATCC 14581 |
| Bacillus mycoides | ATCC 6462 |
| Bacillus sphaericus | ATCC 4525 |
| Bacillus subtilis | ATCC 23857 |
| Bacillus subtilis | DPG 23256 |
| Bacillus thuringiensis | ATCC 10792 |
| Bacillus thuringiensis | ATCC 33679 |
| Bacillus thuringiensis | BGSC 4A1 |
| Brevibacillus brevis | ATCC 8246 |
| Brevibacillus laterosporus | ATCC 9141 |
| Clostridium sporogenes | ATCC 3584 |
| Enterococcus faecalis | ATCC 19433 |
| Escherichia coli | ATCC 13737 |
| Escherichia coli | W3110 |
| Geobacillus stearothermophilus | ATCC 49820 |
| Haemophilus influenzae | ATCC 51907 |
| Listeria monocytogenes | 2432 |
| Pseudomonas aeruginosa | ATCC 17933 |
| Salmonella abaetetuba | ATCC 35640 |
| Serratia marcescens | ATCC 264 |
| Shigella flexneri | ATCC 12022 |
| Shigella sonnei | ATCC 25931 |
| Staphylococcus hominis | ATCC 700236 |
| Staphylococcus aureus | ATCC 25923 |
| Stenotrophomonas maltophilia | ATCC 13637 |
| Streptococcus pneumoniae | ATCC 6308 |
| Streptococcus pyogenes | ATCC 4543 |

### Example 4

### Production of Antibodies that Bind to B. anthracis-Specific Proteins and Production of Vaccines

The B. anthracis Ames strain genome has been sequenced. Ames genome sequences are available at, for example, Genbank Accession No. NC 003997. According to the TIGR database, the nucleotide sequences set forth in SEQ ID NOS. 1, 2, and 3 are parts of nucleotide sequences encoding putative B. anthracis proteins. Specifically, the nucleotide sequence of SEQ ID NO. 1 is found in a gene that may encode a prophage LambdaBa02, FtsK/SpolIIE family protein or a conserved domain protein. The nucleotide sequence of SEQ ID NO. 2 is found in a gene that encodes a putative lantibiotic biosynthesis sensor histidine kinase. The nucleotide sequence of SEQ ID NO. 3 is found in a gene that encodes a putative ABC transporter, ATP-binding protein.

All or part of the nucleic acid sequences of SEQ ID NO. 1, 2, or 3 are placed into an expression vector for expression of the encoded protein as a histidine tagged protein. For example, the pET-21 b+vector (Novogen) or the PGEX-KG vector (Guan and Dixon, Anal. Biochem. 192:262 (1991)) can be used to express the encoded protein as a histidine-tagged protein or a GST-tagged protein. The resulting fusion proteins are then purified via chromatography using Ni²⁺ conjugated chelating Sepharose™ (Pharmacia) for histidine tagged proteins or glutathione Sepharose™ (Pharmacia) for GST-tagged proteins. A sample of the resulting isolated protein is analyzed on an SDS-PAGE gel to test for purity before being solubilized and mixed with Freund's adjuvant for immunizing a host animal.

A mouse or rabbit is then immunized, followed by one or more boosting doses to produce antibodies for testing. For example, mice are initially given 20 μg of purified protein and subsequent booster injections 21 days and 35 days after the initial vaccination. Antibodies are then harvested from serum sample taken from the immunized mice and are analyzed in an ELISA to detect surface binding or in a B. anthracis animal model to measure neutralizing activity. For example, B. anthracis bacteria can be pre-incubated with serum from an immunized animal followed by injection of the pre-incubated bacteria into a susceptible host. The survival of hosts receiving the pre-treated bacteria is then compared with that of hosts receiving untreated bacteria. A greater rate of survival in the hosts receiving the pre-treated bacteria demonstrates the neutralizing activity of the polyclonal antibodies. The same type of ELISAs and neutralization assays are performed on monoclonal antibodies and their genetically engineered counterparts such as chimeric antibodies and humanized antibodies.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supercede any such material. The citation of any references herein is not an admission that such references are prior art to the present invention.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

1. A method for detecting Bacillus anthracis comprising:
(a) providing a sample suspected of containing B. anthracis;
(b) forming a composition comprising nucleic acid from the sample, at least one first primer, and at least one second primer;
(c) amplifying any nucleic acid which the primers in step (b) can amplify; and
(d) detecting B. anthracis by detecting the amplification products of step (c),
(i) wherein at least one primer comprises a nucleotide fragment that is substantially identical to a portion of any of SEQ ID NO. 1, 2, 3, or their complements and wherein the at least one primer specifically binds to B. anthracis DNA or RNA and not to any of B. cereus, B. thuringiensis, and B. subtilis DNA or RNA; and/or (ii) wherein at least one primer comprises at least 12 contiguous nucleotides that are substantially identical to a portion of SEQ ID NO. 1, 2, 3, or their complements.

2. The method of claim 1, wherein each primer comprises at least 12 contiguous nucleotides that are substantially identical to a portion of any of the following six sequences: SEQ ID NO. 1, 2, 3, and their complements.

3. The method of claim 1, wherein the sample comprises non-Bacillus anthracis specific DNA.

4. The method of claim 1, wherein the sample is chosen from the tissue of a host, sputum from a host, soil, water, and air.

5. The method of claim 4, wherein the host is a human or an ungulate.

6. The method of claim 5, wherein the ungulate is a cow or a sheep.

7. The method of claim 1, further comprising extracting nucleic acid from the sample, wherein the nucleic acid is DNA or RNA.

8. The method of claim 1, wherein the nucleic acid is amplified by polymerase chain reaction.

9. The method of claim 1, wherein at least one of said first primers has a nucleotide sequence of SEQ ID NO. 4 or the complement of SEQ ID NO. 4 and at least one of said second primers has a nucleotide sequence of SEQ ID NO. 5 or the complement of SEQ ID NO. 5.

10. The method of claim 1, wherein at least one of said first primers has a nucleotide sequence of SEQ ID NO. 6 or the complement of SEQ ID NO. 6 and at least one of said second primers has a nucleotide sequence of SEQ ID NO. 7 or the complement of SEQ ID NO. 7.

11. The method of claim 1, wherein at least one of said first primers has a nucleotide sequence of SEQ ID NO. 9 or the complement of SEQ ID NO. 9 and at least one of said second primers has a nucleotide sequence of SEQ ID NO. 10 or the complement of SEQ ID NO. 10.

12. The method of claim 1, wherein at least one of said first primers has a nucleotide sequence of SEQ ID NO. 11 or the complement of SEQ ID NO. 11 and at least one of said second primers has a nucleotide sequence of SEQ ID NO. 12 or the complement of SEQ ID NO. 12.

13. The method of claim 1, wherein at least one of said first primers has a nucleotide sequence of SEQ ID NO. 13 or the complement of SEQ ID NO. 13 and at least one of said second primers has a nucleotide sequence of SEQ ID NO. 14 or the complement of SEQ ID NO. 14.

14. The method of claim 1, wherein at least one of said first primers has a nucleotide sequence of SEQ ID NO. 15 or the complement of SEQ ID NO. 15 and at least one of said second primers has a nucleotide sequence of SEQ ID NO. 16 or the complement of SEQ ID NO. 16.

15. The method of claim 1, wherein at least one of said first primers has a nucleotide sequence of SEQ ID NO. 17 or the complement of SEQ ID NO. 17 and at least one of said second primers has a nucleotide sequence of SEQ ID NO. 18 or the complement of SEQ ID NO. 18.

16. The method of claim 1, wherein the at least one first primer or the at least one second primer comprises biotin or digoxigenin.

17. The method of claim 1, wherein the at least one of a first primer or a second primer comprises a detectable label.

18. The method of claim 17, wherein the detectable label is a radioactive label, a fluorescent label, an enzyme, a chemiluminescent moiety, or an ECL moiety.

19. The method of claim 18, wherein the ECL moiety comprises a metal.

20. The method of claim 19, wherein the metal is ruthenium, rhenium, or osmium.

21. The method of claim 18, wherein the ECL moiety is ruthenium(II) tris-bipyridyl ([Ru(bpy)₃]²⁺) or [Ru(sulfo-bpy)₂ bpy]²⁺.

22. The method of claim 17, further comprising incubating the amplified products of step (c) at a temperature sufficient to denature the amplified products and hybridizing the denatured amplified products with an oligonucleotide that can be immobilized on a magnetizable bead before detecting the amplicon.

23. The method of claim 1, wherein the amplified products of step (c) comprises all or a portion of the sequence of SEQ ID NO. 1, 2, 3, or their complements.

24. The method of claim 1, wherein the amplified products of step
(c) is all or a portion of the sequence of SEQ ID NO. 1, 2, 3, or their complements.

25. A method for detecting B. anthracis comprising:
(a) providing a sample suspected of containing B. anthracis;
(b) contacting nucleic acid from the sample with at least one probe under conditions favorable for hybridization; and
(c) detecting B. anthracis in the sample based on the hybridization products of step (b);
(i) wherein at least one probe comprises a nucleotide fragment that is substantially identical to a portion of any of SEQ ID NO. 1, 2, 3, or their complements and wherein the at least one probe specifically binds to B. anthracis DNA or RNA and not to any of B. cereus, B. thuringiensis, and B. subtilis DNA or RNA and/or (ii) wherein at least one probe comprises at least 12 contiguous nucleotides that are substantially identical to a portion of SEQ ID NO. 1, 2, 3, or their complements.

26. The method of claim 25, wherein the probe can hybridize under high stringency conditions with B. anthracis nucleic acid.

27. The method of claim 25, wherein the sample comprises non-Bacillus anthracis specific DNA.

28. The method of claim 25, wherein the sample is chosen from the tissue of a host, sputum from a host, soil, water, and air.

29. The method of claim 28, wherein the host is a human or an ungulate.

30. The method of claim 29, wherein the ungulate is a cow or a sheep.

31. The method of claim 25, further comprising extracting nucleic acid from the sample, wherein the nucleic acid is DNA or RNA.

32. The method of claim 25, wherein the probe is conjugated to a solid support.

33. The method of claim 25, further comprising binding the probe to a solid support after hybridization.

34. The method of claim 25, wherein the probe has a sequence SEQ ID NO. 4, 5, 6, 7, 8, 9, 10, 11, 12, 13, 14, 15, 16, 17, or 18 or a sequence complementary to SEQ ID NO. 4, 5, 6, 7, 8, 9, 10, 11, 12, 13, 14, 15, 16, 17, or 18.

35. The method of claim 25, wherein the at least one probe comprises biotin or digoxigenin.

36. The method of claim 25, wherein the at least one probe comprises a detectable label.

37. The method of claim 36, wherein the detectable label is a radioactive label, a fluorescent label, an enzyme, a chemiluminescent moiety, or an ECL moiety.

38. The method of claim 37, wherein the ECL moiety comprises a metal.

39. The method of claim 38, wherein the metal is ruthenium, rhenium, or osmium.

40. The method of claim 38, wherein the ECL moiety is ruthenium(II) tris-bipyridyl ([Ru(bpy)₃]²⁺) or [Ru(sulfo-bpy)₂ bpy]²⁺.

41. The method of claim 25, wherein the method is a Southern blot.

42. The method of claim 25, wherein the method is a Northern blot.

43. An isolated polynucleotide comprising a section that is (i) substantially identical to a portion of the nucleotide sequence of any of SEQ ID NOS. 1, 2, 3, or their complements, and wherein the polynucleotide specifically binds to B. anthracis DNA or RNA and not to any of B. cereus, B. thuringiensis, and B. subtilis DNA or RNA and/or (ii) substantially identical to at least 12 contiguous nucleotides of any of SEQ ID NOS. 1, 2, 3, or their complements.

44. The isolated polynucleotide of claim 43, wherein the polynucleotide is purified from chromosomal B. anthracis DNA.

45. The isolated polynucleotide of claim 43, wherein the polynucleotide is prepared by recombinant methods.

46. The isolated polynucleotide of claim 45, wherein the recombinant polynucleotide forms a plasmid.

47. The isolated polynucleotide of claim 43, wherein the section is substantially identical to at least 12 contiguous nucleotides of any of SEQ ID NOS. 1, 2, 3, or their complements.

48. A pair of oligonucleotide primers for use in the amplification-based detection of B. anthracis, wherein each primer comprises at least 12 contiguous nucleotides that are substantially identical to a portion of any of the following six sequences: SEQ ID NO. 1, 2, 3, and their complements.

49. The primer pair of claim 48, wherein a first primer has nucleotide sequence SEQ ID NO. 4 or complementary sequence to nucleotide sequence SEQ ID NO. 4 and a second primer has nucleotide sequence SEQ ID NO. 5 or complementary sequence to nucleotide sequence SEQ ID NO. 5.

50. The primer pair of claim 48, wherein a first primer has nucleotide sequence SEQ ID NO. 6 or complementary sequence to nucleotide sequence SEQ ID NO. 6 and a second primer has nucleotide sequence SEQ ID NO. 7 or complementary sequence to nucleotide sequence SEQ ID NO. 7.

51. The primer pair of claim 48, wherein a first primer has nucleotide sequence SEQ ID NO. 9 or complementary sequence to nucleotide sequence SEQ ID NO. 9 and a second primer has nucleotide sequence SEQ ID NO. 10 or complementary sequence to nucleotide sequence SEQ ID NO. 10.

52. The primer pair of claim 48, wherein a first primer has nucleotide sequence SEQ ID NO. 11 or complementary sequence to nucleotide sequence SEQ ID NO. 11 and a second primer has nucleotide sequence SEQ ID NO. 12 or complementary sequence to nucleotide sequence SEQ ID NO. 12.

53. The primer pair of claim 48, wherein a first primer has nucleotide sequence SEQ ID NO. 13 or complementary sequence to nucleotide sequence SEQ ID NO. 13 and a second primer has nucleotide sequence SEQ ID NO. 14 or complementary sequence to nucleotide sequence SEQ ID NO. 14.

54. The primer pair of claim 48, wherein a first primer has nucleotide sequence SEQ ID NO. 15 or complementary sequence to nucleotide sequence SEQ ID NO. 15 and a second primer has nucleotide sequence SEQ ID NO. 16 or complementary sequence to nucleotide sequence SEQ ID NO. 16.

55. The primer pair of claim 48, wherein a first primer has nucleotide sequence SEQ ID NO. 17 or complementary sequence to nucleotide sequence SEQ ID NO. 17 and a second primer has nucleotide sequence SEQ ID NO. 18 or complementary sequence to nucleotide sequence SEQ ID NO. 18.

56. The primer pair of claim 48, wherein at least one primer further comprises a detectable label.

57. The primer pair of claim 56, wherein the detectable label is a radioactive label, a fluorescent label, an enzyme, a chemiluminescent moiety, or an ECL moiety.

58. The primer pair of claim 57, wherein the ECL moiety comprises a metal.

59. The primer pair of claim 58, wherein the metal is ruthenium, rhenium, or osmium.

60. The primer pair of claim 57, wherein the ECL moiety is ruthenium(II) tris-bipyridyl ([Ru(bpy)₃]²⁺) or [Ru(sulfo-bpy)₂ bpy]²⁺.

61. The primer pair of claim 48, wherein at least one primer comprises biotin or digoxigenin.

62. An oligonucleotide probe for use in hybridization-based detection of B. anthracis, wherein the probe comprises at least 12 contiguous nucleotides that are substantially identical to a portion of any of the following six sequences: SEQ ID NO. 1, 2, 3, and their complements, and wherein the probe specifically binds to B. anthracis DNA or RNA and not to any of B. cereus, B. thuringiensis, and B. subtilis DNA or RNA.

63. The oligonucleotide probe of claim 62, wherein the probe is bound to a solid support.

64. The oligonucleotide probe of claim 62, further comprising a detectable label.

65. The oligonucleotide probe of claim 64, wherein the detectable label is a radioactive label, a fluorescent label, an enzyme, a chemiluminescent moiety, or an ECL moiety.

66. The oligonucleotide probe of claim 65, wherein the ECL moiety comprises a metal.

67. The oligonucleotide probe of claim 66, wherein the metal is ruthenium, rhenium, or osmium.

68. The oligonucleotide probe of claim 65, wherein the ECL moiety is ruthenium(II) tris-bipyridyl ([Ru(bpy)₃]²⁺) or [Ru(sulfo-bpy)₂ bpy]²⁺.

69. The oligonucleotide probe of claim 62, wherein the probe comprises biotin or digoxigenin.

70. The oligonucleotide probe of claim 62 further comprising a chemically active group.

71. The oligonucleotide probe of claim 70, wherein the chemically active group is an amino group.

72. The oligonucleotide probe of claim 62, wherein the probe has a sequence SEQ ID NO. 4, 5, 6, 7, 8, 9, 10, 11, 12, 13, 14, 15, 16, 17, or 18 or a sequence complementary to SEQ ID NO. 4, 5, 6, 7, 8, 9, 10, 11, 12, 13, 14, 15, 16, 17, or 18.

73. A kit for the detection of B. anthracis, the kit comprising at least one pair of oligonucleotide primers, wherein each primer comprises at least 12 contiguous nucleotides that are substantially identical to a portion of any of the following six sequences: SEQ ID NO. 1, 2, 3, and their complements, and wherein at least one primer specifically binds to B. anthracis DNA or RNA and not to any of B. cereus, B. thuringiensis, and B. subtilis DNA or RNA.

74. The kit of claim 73, wherein at least one primer pair has nucleotide sequences SEQ ID NO. 4 and SEQ ID NO. 5 or sequences complementary to nucleotide sequences SEQ ID NO. 4 and SEQ ID NO. 5.

75. The kit of claim 73, wherein at least one primer pair has nucleotide sequences SEQ ID NO. 6 and SEQ ID NO. 7 or sequences complementary to nucleotide sequences SEQ ID NO. 6 and SEQ ID NO. 7.

76. The kit of claim 73, wherein at least one primer pair has nucleotide sequences SEQ ID NO. 9 and SEQ ID NO. 10 or sequences complementary to nucleotide sequences SEQ ID NO. 9 and SEQ ID NO. 10.

77. The kit of claim 73, wherein at least one primer pair has nucleotide sequences SEQ ID NO. 11 and SEQ ID NO. 12 or sequences complementary to nucleotide sequences SEQ ID NO. 11 and SEQ ID NO. 12.

78. The kit of claim 73, wherein at least one primer pair has nucleotide sequences SEQ ID NO. 13 and SEQ ID NO. 14 or sequences complementary to nucleotide sequences SEQ ID NO. 13 and SEQ ID NO. 14.

79. The kit of claim 73, wherein at least one primer pair has nucleotide sequences SEQ ID NO. 15 and SEQ ID NO. 16 or sequences complementary to nucleotide sequences SEQ ID NO. 15 and SEQ ID NO. 16.

80. The kit of claim 73, wherein at least one primer pair has nucleotide sequences SEQ ID NO. 17 and SEQ ID NO. 18 or sequences complementary to nucleotide sequences SEQ ID NO. 17 and SEQ ID NO. 18.

81. The kit of claim 73, wherein at least one primer comprises a detectable label.

82. The kit of claim 81, wherein the detectable label is a radioactive label, a fluorescent label, an enzyme, a chemiluminescent moiety, or an ECL moiety.

83. The kit of claim 82, wherein the ECL moiety comprises a metal.

84. The kit of claim 83, wherein the metal is ruthenium, rhenium, or osmium.

85. The primer pair of claim 82, wherein the ECL moiety is ruthenium(II) tris-bipyridyl ([Ru(bpy)₃]²⁺) or [Ru(sulfo-bpy)₂ bpy]²⁺.

86. The kit of claim 73, wherein at least one primer comprises biotin or digoxigenin.

87. A kit for the detection of B. anthracis, the kit comprising at least one oligonucleotide probe, wherein the probe comprises at least 12 contiguous nucleotides that are substantially identical to a portion of the following six sequences: SEQ ID NO. 1, 2, 3, and their complements, and wherein the probe specifically binds to B. anthracis DNA or RNA and not to any of B. cereus, B. thuringiensis, and B. subtilis DNA or RNA.

88. The kit of claim 87, wherein the at least one oligonucleotide probe is bound to a solid support.

89. The kit of claim 87, wherein the at least one oligonucleotide probe can be bound to a solid support.

90. The kit of claim 87, wherein the at least one oligonucleotide probe further comprises a detectable label.

91. The kit of claim 90, wherein the detectable label is a radioactive label, a fluorescent label, an enzyme, a chemiluminescent moiety, or an ECL moiety.

92. The kit of claim 91, wherein the ECL moiety comprises a metal.

93. The kit of claim 92, wherein the metal is ruthenium, rhenium, or osmium.

94. The kit of claim 91, wherein the ECL moiety is ruthenium(II) tris-bipyridyl ([Ru(bpy)₃]²⁺) or [Ru(sulfo-bpy)₂ bpy]²⁺.

95. The kit of claim 87, wherein the at least one oligonucleotide probe further comprises a chemically active group.

96. The kit of claim 95, wherein the chemically active group is an amino group.

97. The kit of claim 87, wherein the oligonucleotide probe has a sequence SEQ ID NO. 4, 5, 6, 7, 8, 9, 10, 11, 12, 13, 14, 15, 16, 17, or 18 or a sequence complementary to SEQ ID NO. 4, 5, 6, 7, 8, 9, 10, 11, 12, 13, 14, 15, 16, 17, or 18.

98. The kit of claim 87, wherein at least one oligonucleotide probe comprises biotin or digoxigenin.

99-106. (canceled)

107. A method for amplifying Bacillus anthracis nucleic acid comprising:
(a) providing a sample of B. anthracis nucleic acid;
(b) forming a composition comprising the nucleic acid and a primer pair;
(c) amplifying the nucleotide sequence between the first primer of the primer pair and the second primer of the primer pair to form an amplicon; and
(d) optionally detecting the amplicon,
(i) wherein at least one primer of the primer pair comprises a nucleotide fragment that is substantially identical to a portion of any of SEQ ID NO. 1, 2, 3, or their complements and wherein the at least one primer specifically binds to B. anthracis DNA or RNA and not to any of B. cereus, B. thuringiensis, and B. subtilis DNA or RNA and/or (ii) wherein at least one primer of the primer pair comprises at least 12 contiguous nucleotides that are substantially identical to a portion of SEQ ID NO. 1, 2, 3, or their complements.

108. The method of claim 107, wherein the nucleic acid is RNA or DNA.

109. The method of claim 107, wherein the at least one of a first primer or a second primer comprises a detectable label.

110. The method of claim 107, wherein the amplicon is all or a portion of the sequence of SEQ ID NO. 1, 2, 3, or their complements.

111. The method of claim 107, wherein a first primer has nucleotide sequence SEQ ID NO. 4 or complementary sequence to nucleotide sequence SEQ ID NO. 4 and a second primer has nucleotide sequence SEQ ID NO. 5 or complementary sequence to nucleotide sequence SEQ ID NO. 5.

112. The method of claim 107, wherein a first primer has nucleotide sequence SEQ ID NO. 6 or complementary sequence to nucleotide sequence SEQ ID NO. 6 and a second primer has nucleotide sequence SEQ ID NO. 7 or complementary sequence to nucleotide sequence SEQ ID NO. 7.

113. The method of claim 107, wherein a first primer has nucleotide sequence SEQ ID NO. 9 or complementary sequence to nucleotide sequence SEQ ID NO. 9 and a second primer has nucleotide sequence SEQ ID NO. 10 or complementary sequence to nucleotide sequence SEQ ID NO. 10.

114. The method of claim 107, wherein a first primer has nucleotide sequence SEQ ID NO. 11 or complementary sequence to nucleotide sequence SEQ ID NO. 11 and a second primer has nucleotide sequence SEQ ID NO. 12 or complementary sequence to nucleotide sequence SEQ ID NO. 12.

115. The method of claim 107, wherein a first primer has nucleotide sequence SEQ ID NO. 13 or complementary sequence to nucleotide sequence SEQ ID NO. 13 and a second primer has nucleotide sequence SEQ ID NO. 14 or complementary sequence to nucleotide sequence SEQ ID NO. 14.

116. The method of claim 107, wherein a first primer has nucleotide sequence SEQ ID NO. 15 or complementary sequence to nucleotide sequence SEQ ID NO. 15 and a second primer has nucleotide sequence SEQ ID NO. 16 or complementary sequence to nucleotide sequence SEQ ID NO. 16.

117. The method of claim 107, wherein a first primer has nucleotide sequence SEQ ID NO. 17 or complementary sequence to nucleotide sequence SEQ ID NO. 17 and a second primer has nucleotide sequence SEQ ID NO. 18 or complementary sequence to nucleotide sequence SEQ ID NO. 18.

118-126. (canceled)
